(19) United States
(12) Patent Application Publication
(10) Pub. No.: US 2010/0081159 A1
(43) Pub. Date: Apr. 1, 2010

(54) Profiling reactive oxygen, nitrogen and halogen species

(76) Inventors: Irina V. Lebedeva, Bronx, NY (US); Wayne Forrest Patton, Dix Hills, NY (US)

Correspondence Address: ENZO LIFE SCIENCES, INC.;c/o ENZO BIOCHEM, INC., 9th Floor, 527 Madison Avenue, New York, NY, 10022-4304, US

(21) Appl. No.: 12/286,103

(22) Filed: Sep. 26, 2008

(51) Int. Cl. C12Q 1/02

(52) U.S. Cl. 435/29

(57) ABSTRACT

This invention provides methods, kits and systems which permit simultaneous profiling of multiple reactive oxygen species (ROS), reactive nitrogen species (RNS) and/or reactive halogen species (RHS) including reactive chlorine species (RCS) and/or reactive bromine species (RBS) through multiplexed fluorescence detection of three or more indicator probes in live cells or subcellular organelles.

### FIELD OF THE INVENTION

This invention relates to the field of free radicals and reactive species in human physiological processes, and more particularly, to the detecting, measuring, profiling and/or monitoring in living cells of such free radicals, e.g., reactive species, including reactive oxygen species (ROS), reactive nitrogen species (RNS) and reactive halogen species (RHS), e.g., reactive chlorine species (RCS) and reactive bromine species (RBS). These free radicals and reactive species are thought to play an important role in many human physiological and pathophysiological processes, including cell signaling, aging, cancer, atherosclerosis, inflammatory diseases, various neurodegenerative diseases and diabetes.

All patents, patent applications, patent publications, scientific articles and the like, cited or identified in this application are hereby incorporated by reference in their entirety in order to describe more fully the state of the art to which the present invention pertains.

### BACKGROUND OF THE INVENTION

Various mammalian enzymes are capable of transferring electrons to molecular oxygen, sequentially forming the one electron-reduction product superoxide (O₂^{•-}) and the two electron reduction product hydrogen peroxide (H₂O₂). These species serve as progenitors for other reactive oxygen species (ROS), including peroxynitrite (ONOO⁻), the hydroxyl radical (⁻OH), hypothiocyanate (HOSCN), lipid peroxides, lipid peroxyradicals, and lipid alkoxyl radicals. A related family of molecules is the reactive nitrogen species (RNS), including nitric oxide (NO), the nitrogen dioxide radical, and the nitrosonium cation. Finally, reactive chlorine species (RCS) and reactive bromine species (RBS), collectively referred to as reactive halogen species (RHS), are also formed under certain biological situations. Specifically, polymorphonuclear leukocytes secrete the heme enzyme myeloperoxidase (MPO) that is, an important weapon in killing and destruction of foreign microorganisms mainly by its halogenating activity. Endogenous hypochlorous acid can contribute to tissue injuries found in inflammatory diseases including respiratory distress, ischemia-reperfusion injury, acute vasculitis, arthritis, gluomerulonephritis and atherosclerotic lesions. At sites of chronic inflammation, activated neutrophils release hydrogen peroxide and the enzyme myeloperoxidase to catalyze the formation of hypochlorous acid. Up to 80% of the hydrogen peroxide generated by activated neutrophils is used to form 20-400 μM hypochlorous acid an hour. A related heme enzyme is the eosinophil peroxidase, released from eosinophils. Owing to its high concentration in biological fluids (100-140 mM Cl⁻, versus 20-100 μM Br⁻ or 1 μM I⁻, Cl— is the major substrate for these peroxidases. It is essential that information-rich methods be developed to quantify the relative levels of various reactive species in living cells and tissues, due to the seminal role that such reactive species play in physiology and pathophysiology.

Ideally, an assay for ROS/RNS detection should be sufficiently sensitive to ensure that measurements are within the linear range of the assay and well above the limits of detection in living cells. Preferably, the assay should be relatively specific for certain ROS/RNS species, at least using physiological or pathophysiological concentrations of the analyte. On the other hand, an assay capable of providing information on global levels of ROS/RNS is also valuable under certain circumstances. Such an assay should be robust, that is to say, meaning that it is applicable to a wide variety of experimental conditions and is comparable among these applications. The assay should be easy to perform and should not require specialized equipment that is normally not available in a standard biomedical laboratory setting. Assays should be designed to monitor the analytes in the context of intact tissues and under proper physiological conditions, rather than in artificial “test tube” situations. The basic approach that comes closest to meeting these fundamental requirements involves the use of certain fluorescent probes. No single fluorescent probe offers, however, the necessarily rich analytical output required to comprehensively provide information on the generation of multiple ROS/RNS analytes.

Several efforts have been made at measuring or detecting ROS species. Among these efforts in which ROS species were measured or detected are peroxide (U.S. Pat. No. 4,269,938), nitric oxide (U.S. Pat. No. 6,569,892), peroxynitrite (US 2007/0082403), superoxide and nitric oxide (U.S. Pat. No. 5,434,085), superoxide (Rothe and Valet, J. Leuk. Biol. 47:440-448 (1990); and U.S. Pat. No. 7,223,864), hydrogen peroxide and superoxide (Maeda, H., Ann. N.Y. Acad. Sci. 1130:149-156 (2008)), and hydrogen peroxide (US 2007/0141658).

Although generally fewer in number, other efforts have been directed at measuring or detecting RNS species. These are summarized as follows. U.S. Pat. No. 5,434,085 provides a method for assaying superoxide or nitric oxide in an aqueous sample, including an initial step of trapping the analytes an emulsion or micellar suspension of a trapping solvent, then reacting the trapped analyte with an appropriate analytical reagent. A flow apparatus for carrying out the method is described that allows continuous introduction of analytical reagent and continuous read-out of the analytical reaction signal, e.g., chemiluminescence intensity.

U.S. Pat. No. 6,569,892 B2 is representative of a family of patents from Dr. Nagano's laboratory relating to fluorescence-based detection of nitric oxide. Other disclosures from this laboratory include U.S. Pat. Nos. 6,441,197; 6,569,892; 6,756,231 and 6,833,386, and two U.S. published applications, 2006/0030054 and 2007/0117211.

The most commonly employed strategy for fluorescence-based detection of NO employs an o-phenylenediamine scaffold, which in the presence of NO and air oxidizes to the corresponding aryl triazole. The electronic differences between the electron-rich diamine and electron-poor triazole groups provide a robust switch for NO detection. A crucial feature contributing to the success of these diamine-based probes is their high selectivity for NO under aerated conditions, as the fluorescent triazole product is not formed by reaction with superoxide, hydrogen peroxide, or peroxynitrite. 1,2-diaminoanthraquinone is not covered by the Nagano family of patents and is commercially available from a number of companies including Molecular Probes/Invitrogen (Eugene, Oreg.), Interchim (Montlucon, FR), Biotium (Hayward, Calif.), to name just a few. The probe was reported to be useful for the analysis of nitric oxide (Heiduschka and Thanos “NO production during neuronal cell death can be directly assessed by a chemical reaction in vivo.” Neuroreport 1998, 9: 4051-4057).

Investigative studies have also been directed towards halogen reactive species, most notably, reactive chlorine species (RCS) and reactive bromine species (RBS). These studies have included the interaction between the production of halogen reactives species and neutrophils (Gaut et al., PNAS 98:11961-11966 (2001)); between halogenating agents and eosinophils (Mayeno et al., JBC 264:5660-5668 (1989)); between brominating intermediates and eosinophils (Henderson et al., JBC 276:7867-7875 (2001)). The role of halogen reactive species in pathology has been postulated, for example, in cancer (Halliwell, B., Biochemical J. 401:1-11 (2007) and Vile et al., Archives of Biochem. And Biophysics 377:122-128 (2000)); and liver cirrhosis (Whiteman et al., Free Radical Biology & Medicine 38:1571-1584 (2005). Cell-based assays are increasingly gaining in popularity in the pharmaceutical industry due to their high physiological relevance. Additional advantages include their ability to predict compound usefulness, evaluate molecular interactions, identify toxicity, distinguish cell type-specific drug effects, and determine drug penetration. Cell-based assays are relevant throughout the drug discovery pipeline, as they are capable of providing data from target characterization and validation to lead identification (primary and secondary screening) to terminal stages of toxicology. Current industry trends of performing drug screening with cell context demand easily monitored, non-invasive reporters. Fluorescent probes fulfill this demand more completely than any other available tools. Requirements for advanced screening assays are driven by the objective of failing candidate compounds early in the drug discovery pipeline. This fundamental approach increases efficiency, reduces costs, and results in shorter time to market for new drugs. In order to fail compounds early, information-rich data for accurate early-stage decision making is required. Such data may be derived by screening compounds in context, that is, by screening in relevant living systems rather than with classical biochemical assays, often incorporating sophisticated imaging platforms, such as high-content screening (HCS) workstations. The industrialization of fluorescent microscopy has led to the development of these high-throughput imaging platforms capable of HCS. When coupled with appropriate fluorescence-based reporter technology, HCS has provided information-rich drug screens, as well as access to novel types of drug targets.

Recent emphasis on multi-color imaging in HCS has created renewed demand for easily measured, non-invasive, and non-disruptive cellular and molecular probes. To date, however, concerted efforts in developing such organic fluorescent probes for ROS/RNS profiling, specifically tailored to working in concert with one another, has been limited in scope. Acceptable probes for cell imaging and analysis need to be minimally perturbing, versatile, stable, easy-to-use, and easy to detect using non-invasive imaging equipment. In the context of the analyses described above, a molecular probe must be able to report upon events in living cells and in real time. Simplicity is of key importance, especially in the context of drug screening.

It would be extremely useful to develop a multiplex system that would allow the investigator to profile different ROS and RNS species and even halogen reactive species (e.g., CRS and BRS) from the same living specimen, and further, to quantify, measure and/or to monitor the level of such species in living cells so as to gauge ongoing physiological and pathophysiological processes.

### SUMMARY OF THE INVENTION

This invention relates to novel combinations of indicator probes, which in concert allow comprehensive profiling of reactive oxygen species (ROS) and reactive nitrogen species (RNS) and reactive halogen species (RHS) (and combinations of these species) in living cells and/or subcellular organelles. In one embodiment, this invention incorporates an indicator probe for global detection or measurement of oxidative and/or nitrative stress and/or halogenating stress, and two or more other indicator probes capable of more restrictive detection of specific ROS or RNS species, without substantial cross-reaction with other ROS or RNS.

The invention also provides methods for measuring three or more indicator probes for profiling the status of ROS, RNS and RHS species, comprising the general steps of contacting the probes mentioned above with the sample, and measuring the signal generated by the probes through reaction between the probes and the targeted ROS and/or RNS and/or RHS present in the sample.

The invention also provides a multi-parameter, high-content screening method for detecting multiple ROS and/or RNS and/or RHS comprising using one or more agents for measuring global ROS and/or RNS and/or RHS and/or one or more agents for detecting specific types of ROS and/or RNS and/or RHS.

The invention also provides a high-throughput method for screening compounds that increase or decrease the production of ROS and/or RNS and/or RHS, employing three or more indicator probes reactive to various ROS or RNS.

The present invention provides more particularly a method for profiling the status of reactive oxygen species (ROS), reactive nitrogen species (RNS) or reactive halogen species (RHS) (or combinations of these species) in living cells or subcellular organelles, or both. This method comprises first (A) providing: (i) at least one sample of living cells or cellular organelles for ROS/RNS/RHS profiling; and (ii) three or more indicator probes. These probes are independently selected from (a) global reactive species probes for detecting or quantifying in living cells or subcellular organelles oxidative stress, nitrative stress, or halogenating stress (and combinations thereof; and (b) selective reactive species probes for detecting specific ROS species, specific RNS species, specific RHS species, and combination of these. Next, the sample of living cells or subcellular organelles (i) are contacted (B) with the three or more indicator probes to generate signals; and the generated signal or signals are measured (C), thereby providing a status profile of specific ROS/RNS/RHS species in the living cells or subcellular organelles (or both) being tested.

The present invention also provides more particularly a method for profiling the status of reactive oxygen species (ROS), reactive nitrogen species (RNS) and/or reactive halogen species (RHS) in living cells or subcellular organelles, or both. In this method, there are first provided (i) at least one sample of living cells or cellular organelles for ROS/RNS/RHS profiling, (ii) three or more indicator probes. These three or more probes are independently selected from (ii) (a) global reactive species probes for detecting or quantifying in living cells or subcellular organelles oxidative stress, nitrative stress, or halogenating stress (and combinations thereof; (ii) (b) selective reactive species probes for detecting ROS species, RNS species, RHS species (and combinations thereof; (iii) (c) one or more inhibitors or scavengers of reactive species generation selected from ROS, RNS, RHS, and combinations thereof; and optionally, (iii) (d) one or more activators, donors or generators of reactive species generation selected from ROS, RNS, RHS, and combinations thereof. In the next step of this method, the sample of living cells or subcellular organelles are initially contacted (B) with (i) with the three or more indicator probes to generate fluorescent signals. The generated signals are measured (C), thereby providing a status profile of specific ROS/RNS/RHS species in the living cells or subcellular organelles under examination.

Also provided by the present invention is a kit in various forms for profiling the status of reactive oxygen species (ROS), reactive nitrogen species (RNS) and/or reactive halogen species (RHS) in living cells or subcellular organelles, or both living cells and subcellular organelles. In packaged combination, the kit comprises (i) three or more indicator probes independently selected from (a) global reactive species probes for detecting or quantifying in living cells or subcellular organelles (or both) oxidative stress, nitrative stress, or halogenating stress (and combinations thereof; and (b) selective reactive species probes for detecting specific ROS species, specific RNS species, or specific RHS species (and combinations thereof); (ii) buffers; and (iii) instructions therefor.

Additionally provided by this invention is a method of quantifying signals from cells, organelles, cell regions or domains of cells of interest (or combinations of any of the foregoing). In the first step of this method, there are provided (A) (i) a sample containing said cells of interest; (ii) at least one solution comprising: (I) three or more indicator probes independently selected from (a) global probes for detecting or quantifying in living cells or subcellular organelles oxidative stress, nitrative stress, or halogenating stress (and combinations thereof); (b) reactive species probes for detecting specific ROS species, specific RNS species, specific RHS species (and combinations thereof; (II) one or more inhibitors of reactive species generation selected from ROS, RNS or RHS (and combinations thereof); and optionally, (III) one or more activators of reactive species generation selected from ROS, RNS, RHS (and combinations thereof); (B) incubating said cells of interest (i) in said solution (ii) to generate signals from cells organelles, cell regions or domains of said cells of interest; and (C) quantifying the generated signal.

Additionally, the present invention provides a method of quantifying signals from cells, organelles, cell regions or domains of cells of interest (or combinations of any of the foregoing). First, there are provided (A) (i) a sample containing the cells of interest; (ii) at least one solution comprising: (I) three or more indicator probes independently selected from: (a) global probes for detecting or quantifying in living cells or subcellular organelles oxidative stress, nitrative stress, or halogenating stress (and combinations thereof; (b) reactive species probes for detecting specific ROS species, specific RNS species, specific halogen species (and combinations thereof); (II) one or more inhibitors of reactive species generation selected from ROS, RNS or RHS (and combinations thereof); and optionally, (III) one or more activators of reactive species generation selected from ROS, RNS, RHS (and combinations thereof). The cells of interest (i) are incubated (B) in said solution (ii) to generate signals from cells organelles, cell regions or domains of the cells of interest, or any of the foregoing. Any generated signal is then quantified (C).

Yet further provided by this invention is a novel system for profiling or monitoring the status of any or all of reactive oxygen species (ROS), reactive nitrogen species (RNS) and reactive halogen species in living cells, subcellular organelles, or both living cells and subcellular organelles. The novel system comprises (i) container means for three or more indicator probes independently selected from (a) global reactive species probes for detecting or quantifying oxidative stress, nitrative stress or halogenating stress (and combinations thereof in living cells or subcellular organelles; and (b) selective reactive species probes for detecting specific ROS species, RNS species; RHS species (and combinations thereof) (ii) other container means for providing optional reagents or components comprising: (c) one or more inhibitors or scavengers of reactive species generation selected from ROS, RNS, RHS (and combinations thereof); and (d) one or more activators, donors or generators of reactive species generation selected from ROS, RNS RHS (and combinations thereof); (iii) means for introducing the probes and the optional reagents or components to a sample of living cells or subcellular organelles; and (iv) instrument, device or means to measure signal generation.

### BRIEF DESCRIPTION OF THE FIGURES

FIG. 1 shows a schematic depiction of ROS/RNS profiling using three fluorogenic probes.

FIG. 2 illustrates in flow chart form ROS/RNS profiling using three fluorogenic probes.

FIG. 3 illustrates specific and selective detection of ROS/RNS production in HeLa cells using triple staining with DAQ/DCFDA/HE and wide-field fluorescence microscopy.

FIG. 4 shows ROS/RNS profiling in HeLa cells using triple staining with DAQ/DCFDA/HE, a set of specific ROS/RNS inducers and inhibitors and method of wide-field fluorescence microscopy.

FIG. 5 demonstrates real time measurements of ROS/RNS levels in HeLa cells using triple staining with DAQ/DCFDA/HE and wide-field fluorescence microscopy.

FIG. 6 are bar graphs that show multiplexed ROS/RNS detection in HeLa cells by triple staining (DAQ/DCFDA/HE) protocol and flow cytometry.

### DESCRIPTION OF THE INVENTION

The invention generally relates to multiplexed analysis using indicator probes suitable for simultaneously monitoring various reactive oxygen species (ROS), and/or reactive nitrogen species (RNS) and/or reactive halogen species (RHS) by wide-field fluorescence microscopy, flow cytometry, confocal microscopy, fluorimetry, high-content cell analysis, cell microarray analysis (positional and nonpositional), high-content cell screening, laser-scanning cytometry and other imaging and detection modalities. The invention relates to employing judiciously selected combinations of cell permeable indicator probes for profiling global ROS, RHS or RNS levels in conjunction with specific classes of ROS/RHS/RNS, such as superoxide (O₂⁻), hypochlorous acid (HOCl) and nitric oxide (NO). Certain probe combinations permit detection of peroxynitrite generation as well, thru monitoring increases in total ROS signal and concomitant decreases in NO signal.

Since no single indicator probe or fluorescent probe can deliver the necessary analytical output required, use of multiple probes should be considered. In order to use them efficiently, multiplexed sets of fluorescent probes must exhibit biological compatibility, optical optimization, and provide insight into the roles of individual, transient ROS and RNS in complex oxidation biology cascades. Biological constraints require that the probes exhibit some measure of water solubility, as well as permeability to extracellular and/or intracellular membranes. The probes should also offer minimal toxicity to living samples. Other requirements for these probes include optical properties tailored toward use in biological environments, including sizable extinction coefficients and quantum yields in aqueous media, and visible or near-IR excitation and emission profiles to reduce or eliminate sample damage and autofluorescence arising from endogenous chromophores or exogenously supplied pathway perturbing agents, such as small molecule ROS activators or inhibitors.

The most commonly employed strategy for fluorescence-based detection of NO employs an o-phenylenediamine scaffold, which in the presence of NO and air oxidizes to the corresponding aryl triazole. The electronic differences between the electron-rich diamine and electron-poor triazole groups provide a robust switch for NO detection. A crucial feature contributing to the success of these diamine-based probes is their high selectivity for NO under aerated conditions, as the fluorescent triazole product is not formed by reaction with superoxide, hydrogen peroxide, or peroxynitrite.

Initially, fluorometric imaging of NO was performed using 2,3-diamino naphthalene (DAN). DAN is poorly soluble in aqueous solution and a UV excitation wavelength (375 nm) is required for imaging, which results in some autofluorescence of endogenous tissue. Due to its nonpolar nature, DAN leaks out of cells after loading. Additionally, DAN exhibits high cellular toxicity. Diaminofluoresceins (DAFs) and diaminorhodamines (DARs) were subsequently synthesized to overcome the problems associated with DAN. In order to solve the problem of sensor leakage from the cells after loading, diacetate derivatives of these dyes were devised. Subsequent hydrolysis of the acetate moieties by intracellular esterases traps the sensors within the cells. However, both reagents have been found to be prone to instability around neutral pH. In an effort to overcome this, 1,3,5,7-tetramethyl-8-(30,40-diaminophenyl)-difluoroboradiaza-s-indacene (TMDA-BODIPY) was synthesized and shown to be photostable and pH independent over a wide range. However, at physiological temperatures TMDA-BODIPY is rapidly protonated, which interferes with its response to NO. Also, TMDA-BODIPY itself is strongly fluorescent, due to two amine moieties as the electron donating groups. When the probe reacts with NO to produce the corresponding triazole, the fluorescence is quenched, making detection of trace levels of NO difficult relative to a corresponding fluorogenic assay format. Finally, other o-phenylenediamine-based probes, including 5,6-diamino-1,3-naphthalene disulfonic acid and 1,2-diaminoanthraquinone (DAQ), have been reported. Certain investigators in the field have discounted such probes, stating that these compounds “ . . . offer no significant improvement over the existing o-diamine based sensors.” (Hilderbrand et al., (2005).

Contrary to the cited conventional wisdom, it has been an unexpected discovery of the present invention that DAQ has superior capabilities relative to many other o-diamine-based NO sensors developed in recent years, particularly with respect to its incorporation into multiplexed fluorogenic profiling assays of ROS and RNS. The reaction of the electron pairs of the free amino groups of non-fluorescent DAQ with NO, in the presence of oxygen, generates a highly fluorescent anthraquinone triazole precipitate having a red emission (emission maximum >580 nm). Peroxynitrite does not react with DAQ and DAQ is stable at neutral pH, as well as at extremes of pH. Additionally, insoluble fluorescent triazole stays in the cells or tissues avoiding leakage problems associated with all other fluorescent probes. The long wavelength emission permits the dye to be multiplexed with other fluorogenic ROS indicators.

Two fluorogenic probes especially suitable for multiplexed analysis of ROS and RNS in conjunction with DAQ are 2′,7′-dichlorofluorescein (DCFH) and dihydroethidium (DHE). DCFH is considered to be a general indicator of ROS, reacting with H₂O₂ (in the presence of peroxidases), ONOO⁻, lipid hydroperoxides, and O₂^{•-}. The diacetate version of the dye is cell permeable, and, after uptake, it is cleaved by intracellular esterases, trapped within the cells, and oxidized to the fluorescent form of the molecule by a variety of ROS. The dye can be detected by strong fluorescence emission at around 525 nm when excited at around 488 nm. Because H₂O₂ is a secondary product of O₂^{•-}, DCFH fluorescence has been used to implicate O₂^{•-} production. The direct reaction of DHE with O₂^{•-} yields a very specific fluorescent product, however, and this requires no conversion to H₂O₂. The product of DHE reaction with O₂^{•-} fluoresces strongly at around 600 nm when excited at 500-530 nm.

### Definitions

By “inhibitor” is meant a substance that decreases the rate of, or prevents, a chemical reaction. An exemplary class of inhibitors are enzyme inhibitors, molecules that bind to enzymes and decrease their activity.

By “scavenger” is meant a chemical substance, added to a mixture or solution, that removes or inactivates unwanted reaction products.

By “activator” is meant a chemical substance that binds to an enzyme and increases its activity. The term activator also refers to a DNA-binding protein that regulates one or more genes by increasing their rate of transcription.

By “inducer” is meant a chemical substance that causes production of another molecule. The term “inducer” also refers to a molecule, usually a substrate of a specific enzyme pathway, that combines with and deactivates an active repressor (produced by a regulator gene); thus allowing an operator gene previously repressed to activate the structural genes controlled by it to resume enzyme production.

By “donor” is meant a chemical substance, added to a mixture or solution, that releases a product over a period of time.

By “generator” is meant a chemical substance, added to a mixture or solution, whose decomposition produces the desired reaction product.

By “fluorescence” is meant the emission of light as a result of absorption of light-emission occurring at a longer wavelength than the incident light.

By “fluorophore” is meant a component of a molecule which causes a molecule to be fluorescent.

By “fluorogenic” is meant a process by which fluorescence is generated. In the context of analytical assays, the term “fluorogenic” refers to a chemical reaction dependent on the presence of a particular analyte that produces fluorescent molecules.

By “indicator probe” is meant a probe that is useful for detecting global or selective reactive species, including reactive oxygen species, reactive nitrogen species and reactive halogen species (Cl or Br), and which is further capable of providing a detectable or quantifiable signal.

By “fluorescent probe” is meant an entity, be it a small organic fluorophore, a fluorescent protein, a nanoparticle or a quantum dot, that is useful for monitoring a chemical or biological event or environment.

Other additional aspects about these terms and definitions may become apparent when reading further descriptions of the present invention.

### Selectivity Profiles of Fluorescent Probes for Various ROS/RNS:

Numerous fluorescent probes have been developed over the years for the purpose of monitoring the production of ROS or RNS in solution, cells, tissues or even whole organisms, as summarized in Table one. Often, a probe has been designated as being specific to one particular analyte, but in fact it may display some selectivity for a particular analyte but also may cross-react with others to some extent. For example, DCFH, 2-[6-(4′-hydroxy)phenoxy-3H-xanthen-3-on-9-yl]benzoic acid (HP F) and 2-[6-(4′-amino)phenoxy-3H-xanthen-3-on-9-yl]benzoic acid (APF) are fluorescent probes for the detection of ROS (Setsukinai et al, 2003).

TABLE 1
Various fluorescent probes developed for detection of ROS or RNS

| | Excitation | Emission | | |
|---|---|---|---|---|
| | Maximum | Maximum | | |
| Fluorescent probe | (nm) | (nm) | Selectivity | Reference |
| 2-(2-pyridyl)- | 377 | 528 | Superoxide | Tang et al, Anal Biochem, 2004; 326: 176-182 |
| benzothiazoline | | | | |
| Amplex Red | 563 | 587 | hydroperoxides | Zhou et al., Anal Biochem 253: 162, 1997 |
| APF | 488 | 515 | hydroxyl radical, | Setsukinai et al J. Biol. Chem 278, 5,. 3170-3175, 2003 |
| | | | peroxynitrite, | |
| | | | hypochlorite | |
| | | | anion | |
| Bis-2,4- | 488 | 525 | Superoxide | Maeda H et al, J Am Chem Soc, 2005; 127: 68-69 |
| dinitrobenzenesulfonyl | | | | |
| fluoresceins | | | | |
| BODIPY FL EDA | 488 | 525 | Lipid peroxides | Franco et al, J Biol Chem. 2007; 282: 30452-30465 |
| C₁₁-BODIPY | 510 | 595 | ROO⁻, RO⁻, HO⁻ | |
| CCA/SECCA | 350/395 | 450 | Hydroxyl radical | Makrigiorgos GM et al, Int J Radiat Biol, 1993, 63: 445-458 |
| Copper (II) | 488 | 525 | Nitric Oxide | Lim MH et al. Nat Chem Biol 2006; 2: 375-380 |
| Fluorescein | | | | |
| CsPA | 325/355 | 440/40 | Lipid peroxides | Makrigiorgos GM et al, J Biochem Biophys Methods. 1997; |
| (cis-parinaric acid) | | | | 35: 23-25 |
| DAC (diaminocyanine) | 750 | 790 | Nitric oxide | Sasaki E et al, J Am Chem Soc 2005; 127: 3684-3685 |
| DAF-2 | 495 | 515 | RNS | Rathel et al Biol. Proced. Online; 5(1): 136-142, 2003 |
| DAMBO-P^{H} | 521 | 537 | Nitric oxide | Gabe et al J. Am Chem. Soc. 126, 3357-3367, 2004 |
| DAQ | 488 | >580 | Nitric oxide | Galindo, Photochem. Photobiol. Sci., 7, 126-130, 2008 |
| DAR-4M | 560 | 575 | RNS | Lacza et al, Journal of Pharmacological and Toxicological |
| | | | | Methods 52 335-340, 2005 |
| DCFDA | 488 | 525 | ROS | Halliwell and Whiteman British Journal of Pharmacology |
| | | | | 142, 231-255, 2004 |
| DHE | 500-530 | 600 | Superoxide | Halliwell and Whiteman British Journal of Pharmacology |
| | | | | 142, 231-255, 2004 |
| DHR | 500 | 536 | Peroxide, HOCl, | Halliwell and Whiteman British Journal of Pharmacology |
| | | | ONOO⁻ | 142, 231-255, 2004 |
| Dihydrocalcein | 494 | 517 | Peroxynitrite, | Keller et al. Free Radical Res 38 (12): 1257-1267, 2004 |
| | | | hydroxyl | |
| | | | radicals, | |
| DMA | 375 | 436 | Singlet oxygen | Corey EJ and Taylor WC, J Am Chem Soc, 1964; 86: 388$$-3882 |
| DMAX | 495 | 515 | Singlet oxygen | Tanaka k et al, J Am Chem Soc, 2001, 123: 2530-2536 |
| Dobz derivatives | 348 | 440 | hydroperoxide | Lo L-C and Chu C-Y, Chem Commun, 2003: 2728-2729 |
| DPAX | 495 | 515 | Singlet oxygen | Umezawa N et al, Angew Chem Int Ed, 1999; 38: 2899-2901 |
| (9-[2-(3-carboxy-9,10- | | | | |
| diphenyl)anthryl]-6- | | | | |
| hydroxy-3H-xanthen- | | | | |
| 3-one) | | | | |
| DPBF | 410 | 455 | Superoxide, | Ohyashiki T et al, Biochem Biophys Acta, 1999; 1421: 131-139 |
| (1,3- | | | singlet oxygen | |
| diphenylisobenzo- | | | (fluorescence | |
| furan) | | | decrease) | |
| DPPEA-HC | 351 | 380 | peroxides | Soh N et al, Bioorg Med Chem, 2005; 13: 1131-1139 |
| DPPEC | 355/40 | 460/25 | Hydroxyl radical | Soh N et al, Anal. Sci. 2008; 24: 293-296 |
| DPPP (Diphenyl-1- | 351 | 380 | Hydroperoxide, | Akasaka K et al, Anal lett, 1987; 20: 731-745, 797-807 |
| pyrenylphosphine) | | | peroxides | |
| FL₅ | 499 | 520 | Nitric oxide | Lim et al Nat Chem Biol. 2(7): 375-80, 2006 |
| HKOCI-1 | 520 | 541 | Hypochlorite | Sun et al, Org. Lett., 10 (11), 2171-2174, 2008. |
| Homovanilic acid | 312 | 420 | hydroperoxide | Ruch et al., J Immunol Meth, 1983; 63: 347-357 |
| HPF | 488 | 515 | hydroxyl radical, | Setsukinai et al J. Biol. Chem 278, 5,. 3170-3175, 2003 |
| | | | peroxynitrite, | |
| HySOX | 552 | 575 | Hypochlorite | Kenmoku et al. J. Am. Chem. Soc., 129, 7313-7318, 2007 |
| Metal-Based Turn-On | dif | dif | Nitric Oxide | Lim MH and Lippard SJ. Acc.Chem.Res. 2007; 40: 41-51 |
| Fluorescent probes | | | | |
| MitoPY1 | 510 | 528 | Hydrogen | Dickinson and Chang J. Am Chem. Soc. 2008, 130, 9638-9639 |
| | | | peroxide | |
| Mito-SOX | 396, 510 | 580 | Superoxide | Robinson et al, PNAS 103 (41), 15038-15043, 2006 |
| MitoTracker Orange | 550 | 574 | peroxides | Whitaker et al, Biochem.Biophys. Res.Commun. 1991, 17$$: |
| (Dihydrotetramethyl- | | | | 387-393. |
| rosamine) | | | | |
| NBD-CI | 470 | 550 | superoxide | Olojo RO et al, Anal Biochem, 2005; 339: 338-344 |
| (4-chloro-7- | | | | |
| nitrobenzo-2-oxa-1,3- | | | | |
| diazole) | | | | |
| NFDS-1 | 602 | 662 | Hydrogen | Xu et al Chem. Commun., 5974-5976, 2005 |
| | | | peroxide | |
| Pentafluorobenzene- | 488 | 525 | hydroperoxide | Maeda H et al, Angew Chem Int Ed, 2004; 43: 2389-2391 |
| sulfonyl fluorescein | | | | |
| Peroxifluor-1 | 488 | 525 | Hydroperoxide | Chang MCY, J Am Chem Soc, 2004; 126: 15392-15393 |
| | | | (very sensitive) | |
| Peroxycrimson-1 | 550 | 575 | Hydrogen | Miller et al, Nat Chem Biol. 3(5): 263-7, 2007 |
| | | | peroxide | |
| Peroxygreen-1 | 450 | 520 | Hydrogen | Miller et al, Nat Chem Biol. 3(5): 263-7, 2007 |
| | | | peroxide | |
| Peroxyresorufin-1 | 543 | 548-644 | Hydrogen | Miller et al, J Am Chem Soc. 2005; 127: 16652-16659 |
| | | | peroxide | |
| o-Phenylenediamine | Various | | Nitric oxide | Plater et al, J Chem Soc Perkin Trans. 2001; 1: 2553-2559 |
| derivatives | fluorophores | | | |
| scopoletin | 360 | 460 | hydroperoxides | Freedman JE et al., J Clin Invest, 1996, 97: 979-987 |
| Spy-HP | 524 | 535 | hydroperoxides | Soh N et al, Bioorg Med Chem Lett. 2006; 16: 2943-2946 |
| Rhodamine | 540 | 575 | Nitric oxide | Zhen H et al, Org Lett. 2008; 10: 2357-2360 |
| spirolactam | | | | |
| SNAPF | 625 | 735 | Hypochlorite | Shepherd, et al Chem. Biol. 14, 1221-1231, 2007 |
| Singlet Oxygen | 504 | 525 | Singlet Oxygen | Flors C et al. J Exp Bot. 2006; 57: 1725-1734. |
| Sensor Green | | | | |
| Terephtalic acid | 326 | 432 | Hydroxyl radical | Qu X et al, Photochem Photobiol, 2000; 71: 307-313 |
| TMDA BODIPY | 500 | 530 | Nitric Oxide | Zhang X et al, Anal Chim Acta. 2003; 481: 101-108 |

To summarize, in many but not all cases, it would be inappropriate to assume that the various indicator probes detect a specific oxidizing species within cells, such as hydroxide, peroxide, hypochlorous acid or nitric oxide. Rather, these probes often detect a broad range of oxidizing reactions that may be increased during intracellular oxidative stress. The promiscuity of many of the fluorescent probes presents an analytical challenge, as it is commonly believed that each species of ROS is likely to have a specific role in living cells. If novel indicator probes were available that allowed comprehensive detection of a variety of ROS/RNS but also provided selective detection of particular reactive species, such probes would contribute greatly to the elucidation of the roles of individual ROS/RNS in living cells. Such probes would also permit high resolution spatiotemporal tracking of the generation of specific ROS. In certain situations, the combination of two different probes with different selectivity profiles for various ROS/RNS has been demonstrated. Given the large number of potential reactive species generated in a cell, however, duplex dye analysis still does not provide a rich enough analytical readout for full characterization of oxidative stress.

Combinations of three or more fluorophores potentially provide a better solution to ROS/RNS profiling. Conventional ROS/RNS detection using a single fluorogenic probe, though allowing the researcher to test many samples at once, can test only one type of ROS/RNS in a single test. This makes the simultaneous testing of multiple analytes unwieldy with respect to time, labor, reagents and sample volume. Together with the importance of profile generation when exploring the complexity and range of ROS/RNS usually found in a biological context, these factors render this type of analysis especially in acute need of multiplexing.

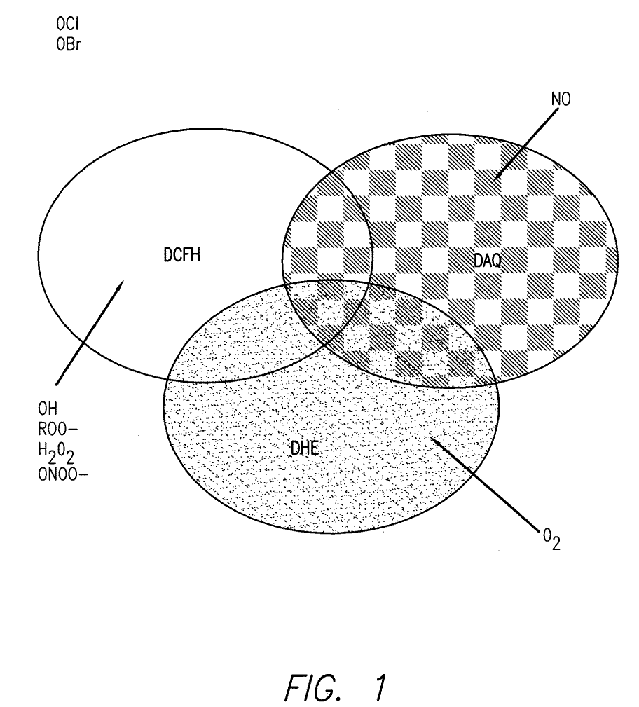

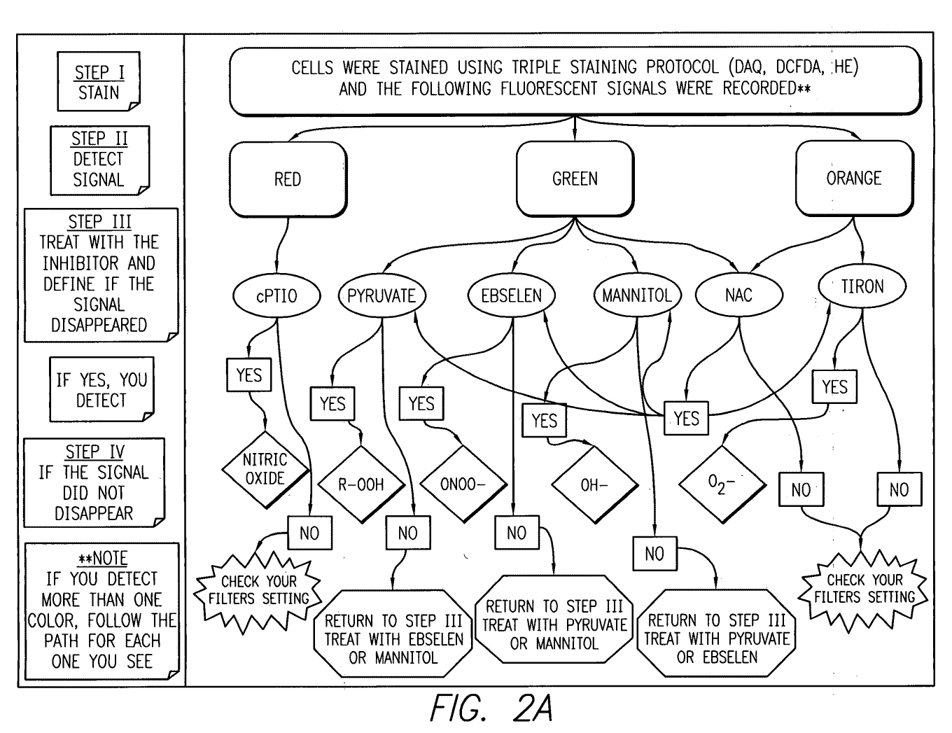

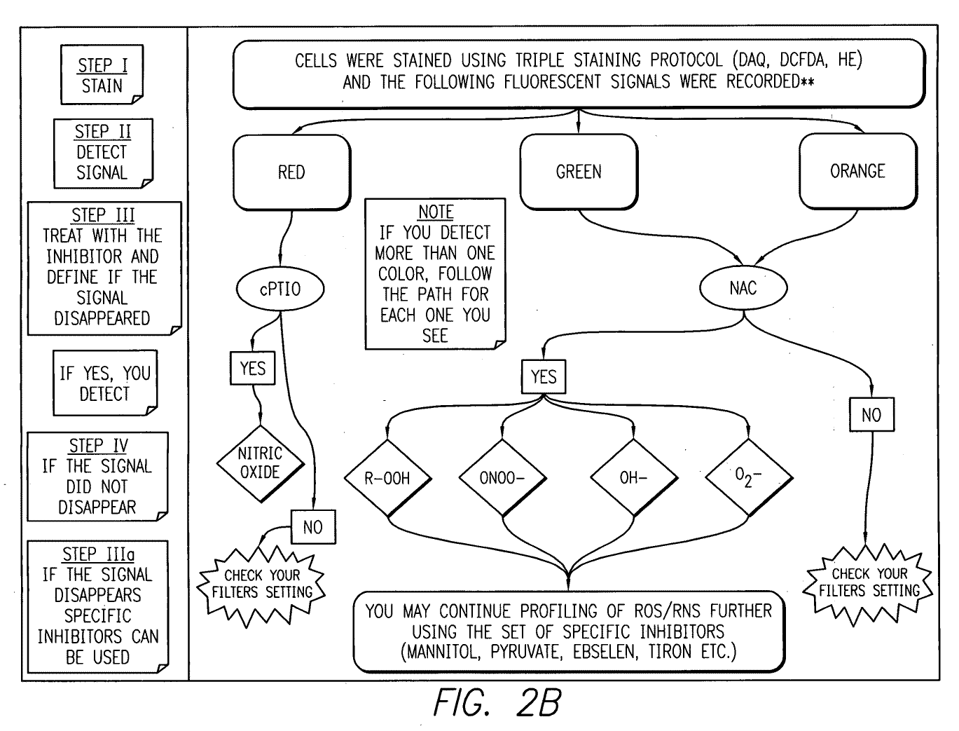

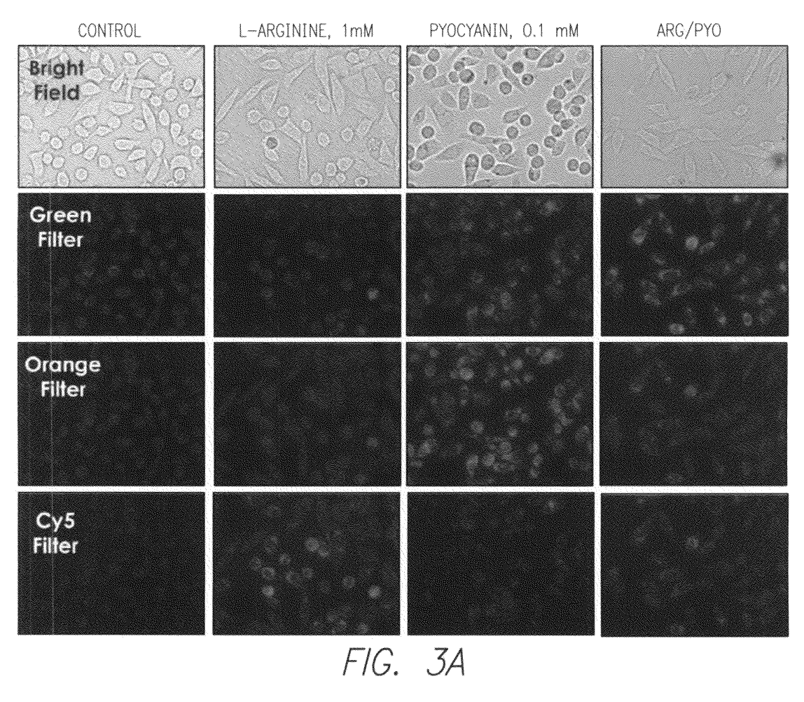

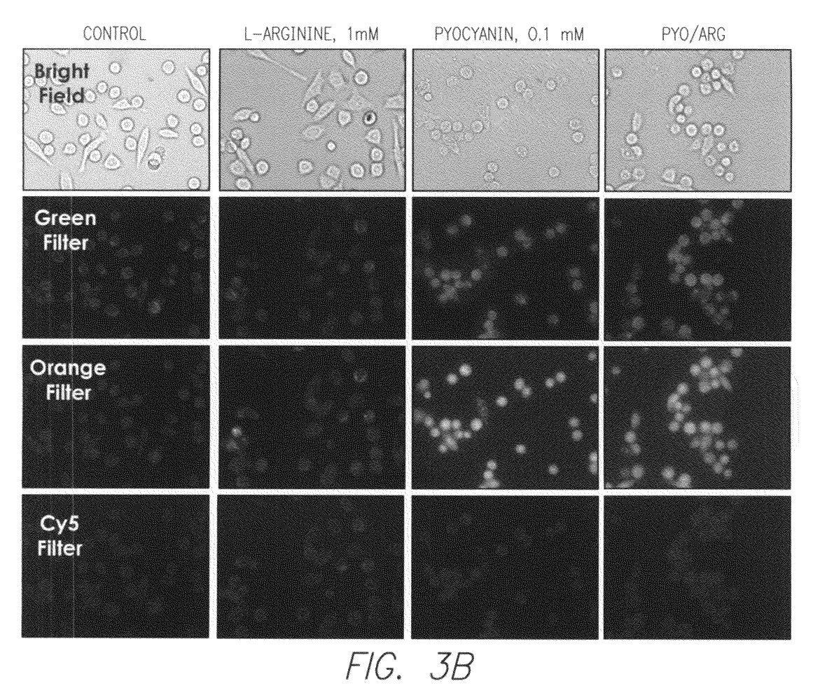

As an example of the utility of this approach, a three-parameter assay according to the present invention is described in FIG. 1. As depicted in FIG. 1, utilizing a general ROS/RNS probe, such as DCFH in conjunction with a highly selective superoxide probe, such as DHE and a highly selective nitric oxide probe, such as DAQ, provides a richer analytical output than any of the probes by themselves or combined in a binary fashion with one another (see the flow chart information in FIGS. 2A and 2B). As indicated in FIG. 2A, total ROS/RNS levels can simultaneously be monitored in conjunction with superoxide and nitric oxide levels using this assay. As summarized in FIG. 2A, DAQ indicates nitric oxide generation in the cell system, DHE indicates superoxide generation and the reaction of nitric oxide and superoxide to generate peroxynitrite is detected by DCFH. The system is relatively insensitive to certain ROS, such as hypochlorite (⁻OCl) and hypobromite

(⁻OBr). While DCFH detects a plethora of ROS/RNS, the analytical confidence in measuring peroxynitrite generation using this multi-parametric assay can be increased through employment of appropriate controls that incorporate relatively selective inhibitors of various reactive species, such as mannitol to block OH generation, sodium pyruvate to block H₂O₂ generation, and ebselen (2-phenyl-1,2-benzisoselenazol-3[2H]-one) to block peroxynitrite generation. Additionally, a fourth or even a fifth fluorogenic probe may be included to further refine analysis, employing regions of the visible or IR light spectrum not already occupied by the other three fluorophores. When multiple fluorogenic probes in the outlined assay are activated, there is an indication that multiple reactive species have been produced by the treatment. For example, pyocyanin generates both hydroxide and superoxide, causing both DCFH and DHE fluorescence.

A walk-through the information depicted in FIG. 2A should also be illuminating. The scheme in FIG. 2A depicts a process for profiling ROS/RNS in live cells that consists of the following steps:

1. Loading of the cells with desired probes (e.g. DAQ, DCFDA and HE)

2. Treatment with the inducer/donor

3. Observation under fluorescence microscope using appropriate filter sets.

If red signal is registered (compared to untreated cells), it may indicate NO production. To confirm that option, control cells should be pre-treated with cPTIO (specific NO scavenger and general NOS inhibitor). If the signal disappeared after pre-treatment with cPTIO, NO production is established. If red signal still can be detected in cPTIO treated cells, filter settings should be checked and corrected to avoid spectra overlapping.

If orange signal is registered (compared to untreated cells), it may indicate superoxide production. To confirm that option, control cells should be pre-treated with NAC (general ROS inhibitor/scavenger) and/or Tiron (specific superoxide scavenger). If the signal disappeared after pre-treatment with NAC or Tiron, superoxide production is established. If orange signal still can be detected in NAC/Tiron treated cells, filter settings should be checked and corrected to avoid spectra overlapping.

If green signal is registered (compared to untreated cells), it may indicate high level of oxidation stress in general with production of peroxide/peroxynitrite/hydroxyl radicals. To confirm that option, control cells should be pre-treated with NAC (general ROS inhibitor/scavenger) first. If green signal still can be detected in NAC treated cells, filter settings should be checked and corrected to avoid spectra overlapping. If the signal disappeared after pre-treatment with NAC, high level of oxidation stress in general with production of peroxide/peroxynitrite/hydroxyl radicals is established. Further profiling of ROS will include pretreatment of the cells with specific ROS inhibitors/scavengers. Recommended are using pyruvate (for peroxides), mannitol (for hydroxyl radicals) and ebselen (specific peroxynitrite scavenger).

Positive control treatments inducing specific ROS/RNS types is highly recommended in all cases. Concentrations of inducers and inhibitors should be optimized for each particular cellular system. Note that most of inhibitors/scavengers at certain concentrations are able to induce oxidative stress themselves due to changes they made in the redox status of the cell.

If more than one color is detected compared to the untreated cells, one should follow the path for each positive signal you see with corresponding inducers/inhibitors.

The depiction in FIG. 2B represents a continuation of additional information to that provided in FIG. 2A, particularly with respect to inhibitors and scavengers (pyruvate, ebselen, Tiron and mannitol) that may be employed to detect specific reactive species in accordance with the present invention and method

### Multiplexed Analysis Using Combinations of Redox-Sensitive Fluorescent Proteins and Fluorogenic ROS/RNS Probes.

The green fluorescent protein from Aequorea victoria has two widely separated excitation maxima whose ratio depends upon the structure of the molecule and hence can depend on external environmental conditions. Redox-sensitive variants of the green fluorescent protein (roGFPs) have been developed that allow “real-time” monitoring of the redox status of cellular compartments by fluorescence excitation ratiometry (Dooley et al, 2004). The GFP variant is responsive to hydrogen peroxide and superoxide. Conversion of roGFP from the reduced to oxidized state leads to a ratiometric increase in fluorescence excitation at the 395-nm peak with an accompanying decrease in excitation at 475 nm. Expression of roGFP in the cytosol and mitochondria of mammalian cells provides effective indicators of the ambient redox potential, as perturbed by exogenous oxidants and reductants, as well as by physiological redox changes.

In an analogous manner, a genetically encoded, highly specific fluorescent probe for detecting hydrogen peroxide inside living cells has also been described (Belousov et al., 2006). Referred to as HyPer, this probe consists of circularly permuted yellow fluorescent protein (cpYFP) inserted into the regulatory domain of the prokaryotic H₂O₂-sensing protein, OxyR.

Much like DCFA, roGFP can be considered a nonselective indicator of ROS, while much like Peroxycrimson-1, HyPer is a high selective indicator for H₂O₂. Different combinations of the redox-sensitive proteins and fluorogenic ROS/RNS organic probes can achieve the intent of the invention to provide a comprehensive analytical readout of ROS/RNS in living cells. For example, cells expressing roGFP and HyPer that are treated with DAQ can provide an analytical readout that is analogous to a combination of DCFA, Peroxycrimson-1 and DAQ.

### Instrumentation Settings for Multiplexed Analysis of ROS/RNS

Although linear unmixing systems should provide the ability to distinguish among large numbers of different fluorophores with partially overlapping spectra, even with a simpler optical setup in wide-field microscopy, it is possible to clearly distinguish among three or more dyes of the present invention. For instance, using appropriate filter sets, one may simultaneously image DCFH, DHE and DAQ described in the present invention, with minimal spectral cross-talk. One possible filter set combination appropriate for performing such an experiment is summarized in Table 2.

TABLE 2
Possible filter set combination for 3-parameter imaging measurements
with various fluorogenic ROS/RNS probes.

| | Analyte | Excitation filter | Emission filter |
|---|---|---|---|
| Fluorogenic probe | measured | (nm) | (nm) |
| DCFH | Various | 490 | 525 |
| | ROS/RNS | | |
| DHE | Superoxide | 550 | 620 |
| DAQ | Nitric oxide | 650 | 670 |

In addition, an appropriately selected fourth probe may be incorporated in the multiplexed analysis, for example, by using a filter combination as outlined in Table 3.

TABLE 3
Possible emission filter set combination for 4-parameter imaging
measurements with various fluorogenic ROS/RNS probes.

| | Analyte | Excitation filter | Emission filter |
|---|---|---|---|
| Fluorogenic probe | measured | (nm) | (nm) |
| DCFH | Various | 490 | 525 |
| | ROS/RNS | | |
| DHE | Superoxide | 550 | 620 |
| DAQ | Nitric oxide | 650 | 670 |
| DPPEC | hydroxyl | 355 | 460 |
| | radicals | | |

In the above example, DPPEC, 1,2-dipalmitoylglycerophosphorylethanolamine labeled with coumarin, is a phospholipid-linked coumarin probe that senses lipid radicals in membranes (Soh et al, 2008).

### More Examples of Reactive Species Scavengers, Inhibitors, Activators, Donors and Generators

Listed below in Table 4 is a more comprehensive list of the various components contemplated for use in the present invention for profiling or monitoring reactive species of oxygen and nitrogen. The list below (Table 4) is not intended to be exhaustive or limiting as there are other scavengers, inhibitors, activators, donors and generators which could be used in accordance with the present invention.

TABLE 4
Examples of reactive species scavengers, inhibitors, activators, donors and
generators.

| Agent | Effect |
|---|---|
| NO Scavengers/NOS inhibitors: | |
| 3-Bromo-7-Nitroindazole | Non-selective NOS inhibitor |
| 5,5-dimethyl-1-pyrroline N-oxide | NO-scavenger |
| 7-Nitroindazole | Non-selective NOS inhibitor |
| Carboxy-PTIO (cPTIO) | Nitric oxide (NO) scavenger and NOS inhibitor |
| Cyanidin chloride | Nitric oxide (NO) scavenger |
| Cyclosporin A | Inhibits nitric oxide (NO) synthesis |
| FeTMPyP (Iron (III) tetrakis(N-methyl-4′- | Synthetic porphyrin complexed with iron which acts as a |
| pyridyl)porphyrin•5Cl) | peroxynitrite decomposition complex. |
| Fusidic acid | Suppresses nitric oxide (NO) synthesis |
| Iromycin A | Inhibitor of nitric oxide synthases (NOS) showing |
| | selectivity for eNOS (NOS III) versus nNOS (NOS I) |
| L-NAME | Competitive inhibitor of NOS |
| L-NMMA | Non-specific NO-inhibitor |
| L-NNA | Competitive inhibitor of NOS (used preferably in in vivo |
| | studies) |
| MEG, sodium succinate | Inhibitor of inducible nitric oxide synthase (iNOS; NOS II). |
| (Mercaptoethylguanidine, sodium succinate) | Peroxynitrite scavenger. |
| Pelargonidin chloride | Nitric oxide scavenger. Antioxidant flavonoid. |
| PTIO (2-Phenyl-4,4,5,5-tetramethylimidazoline- | Nitric oxide (NO) scavenger. |
| 1-oxyl-3-oxide) | |
| Rutin | Nitric oxide (NO) scavenger. |
| Trolox ® (6-Hydroxy-2,5,7,8- | Prevents peroxynitrite-mediated oxidative stress. |
| tetramethylchroman-2-carboxylic acid) | |
| Wogonin | Suppresses the release of nitric oxide (NO) by inducible |
| | nitric oxide synthase (iNOS; NOS II), antioxidant |
| NO donors/generators: | |
| DETA NONOate | Nitric oxide (NO) donor. Induces apoptosis in macrophages |
| Diethylamine NONOate (DEA/NO; DEA | Nitric oxide (NO) donor |
| NONOate) (CAS 56329-27-2) | |
| Angeli's Salt (Hyponitric acid) | Nitric oxide (NO) donor. |
| BNN3 (N,N′-Dimethyl-N,N′-dinitroso-p- | Cell permeable, photolabile NO donor |
| phenylenediamine) | |
| Concanamycin A | Induces nitric oxide (NO) production |
| DD1 (3-Bromo-3,4,4-trimethyl-3,4- | Cell permeable thiol-induced nitric oxide donor |
| dihydrodiazete 1,2-dioxide) | |
| DD2 (3-Bromo-4-methyl-3,4-hexamethylene- | Cell permeable thiol-induced nitric oxide donor |
| 3,4-dihydrodiazete 1,2-dioxide) | |
| DEA/NO | Nitric oxide (NO) donor. |
| (2-(N,N-Diethylamino)-diazenolate-2-oxide) | |
| Dephostatin | Protein S-nitrosylating reagent |
| DETA NONOate (Diethylenetriamine | Nitric oxide (NO) donor. Induces apoptosis in macrophages |
| NONOate) (CAS 146724-94-9) | |
| DPTA NONOate | Nitric oxide (NO) donor |
| Fructose-SNAP-1 | Nitric oxide (NO) donor with increased cytotoxicity compared |
| | to SNAP |
| GEA 3162 | Water soluble nitric oxide (NO) donor |
| GEA 5024 | Water soluble and stable nitric oxide (NO) donor. |
| GEA 5583 | Stable nitric oxide releasing compound that is orally |
| | absorbed in rats. |
| Glyco-SNAP-1 | Highly water soluble nitric oxide (NO) donor |
| Glyco-SNAP-2 | Highly water soluble nitric oxide (NO) donor |
| GSNO | Carrier of nitric oxide (NO) |
| Isosorbide dinitrate | Nitric oxide (NO) donor. |
| L-Arginine | Physiological precursor for the formation of nitric oxide |
| | (NO) by nitric oxide synthase (NOS). Enhances the release |
| | of NO. |
| MAHMA NONOate | Nitric oxide (NO) donor. |
| Molsidomine | Long acting antianginal drug that is enzymatically converted |
| | in the liver to yield the active metabolite SIN-1 (NO donor) |
| N-Cyclopropyl-N′-hydroxyguanidine | Selective substrate for nNOS (NOS I) |
| NOC-12 (1-Hydroxy-2-oxo-3-(N-ethyl-2- | Nitric oxide (NO) donor. |
| aminoethyl)-3-ethyl-1-triazene) | |
| NOC-5 (1-Hydroxy-2-oxo-3-(3-aminopropyl)-3- | Nitric oxide (NO) donor. |
| isopropyl-1-triazene) | |
| NOC-7 (1-Hydroxy-2-oxo-3-(N-3-methyl- | Nitric oxide (NO) donor. |
| aminopropyl)-3-methyl-1-triazene) | |
| NO-Indomethacin (NCX 2121) (CAS 301838- | Nitric oxide (NO) donor. |
| 28-8) | |
| NOR-1 ((±)-(E)-Methyl-2-[(E)-hydroxyimino]-5- | Nitric oxide (NO) donor. |
| nitro-6-methoxy-3-hexeneamide) | |
| NOR-2 ((±)-(E)-Methyl-2-[(E)-hydroxyimino]-5- | Nitric oxide (NO) donor. |
| nitro-3-hexenamide) | |
| NOR-3 (FK409; (±)-(E)-Ethyl-2-[(E)- | Nitric oxide (NO) donor. |
| hydroxyimino]-5-nitro-3-hexeneamide) | |
| NOR-4 (FR 144420; (±)-(E)-Ethyl-2-[(E)- | Nitric oxide (NO) donor. |
| hydroxyimino]-5-nitro-3- | |
| hexenecarbamoylpyridine | |
| NOR-5 ((±)-2-((E)-4-Ethyl-3[(Z)- | Nitric oxide (NO) donor. |
| hydroxyimino]6-methyl-5-nitro-heptenyl)-3- | |
| pyridinecarboxamide) | |
| PAPA NONOate | Nitric oxide (NO) donor. |
| Peroxynitrite•tetramethylammonium | This formulation of peroxynitrite has a low nitrite content |
| | (~1%), no hydrogen peroxide. |
| Piloty's Acid (benzenesulphonydroxamic acid) | Nitric oxide (NO) donor |
| (CAS 599-71-3) | |
| PROLI NONOate | Nitric oxide (NO) donor. |
| SIN-1 chloride | Using molecular oxygen it generates superoxide and nitric |
| | oxide (NO) that together spontaneously form peroxinitrite. |
| SIN-1A/γCD Complex | Physiologically active nitric oxide (NO) releasing agent. |
| SNAP | Nitric oxide (NO) donor and a source of NO in vivo. |
| S-Nitrosocaptopril | Angiotensin-converting enzyme (ACE) inhibitor. Inhibitor of |
| | platelet aggregation. Its activity may depend on the |
| | homolytic cleavage of the S—N bond under physiological |
| | conditions, yielding nitric oxide (NO) and the parent |
| | compound, captopril |
| S-Nitroso-L-glutathione GSNO (CAS 57564-91- | S-nitrosothiol NO donor |
| 7) | |
| Sodium nitroprusside | Nitric oxide (NO) donor. |
| Spermine NONOate | Nitric oxide (NO) donor. |
| Spermine NONOate (CAS 136587-13-8) | Nitric oxide (NO) donor |
| Streptozotocin | N-nitroso-containing antibiotic, acting as a nitric oxide (NO) |
| | donor. |
| Sulfo-NONOate | Dissociates to sulfate and nitrous oxide in a pH-dependent |
| | manner. |
| V-PYRRO/NO | Liver-selective nitric oxide (NO) donor. |
| β-Gal NONOate | Nitric oxide (NO)donor. |
| β-Gal-NONOate (CAS 357192-78-0) | Nitric oxide (NO) donor |
| Free radical scavengers/inhibitors: | |
| (Z)-4-Hydroxytamoxifen | Has antioxidant properties. Intramembranous inhibitor of |
| | lipid peroxidation. |
| 3,5-Di-O-caffeoylquinic acid | Antioxidant. |
| 4-Amino-TEMPO, free radical | Free radical trap. |
| Allicin | Antioxidant |
| Angoroside C | Shows potent antioxidative activity in reducing the oxidized |
| | OH adducts of dAMP and dGMP. |
| Apigenin | Antioxidant flavonoid. |
| Astaxanthin | Extremely potent antioxidant. |
| Bakuchiol | Antioxidant. Inhibitor of mitochondrial lipid peroxidation. |
| | Inhibitor of inducible nitric oxide synthase (iNOS; NOS II) |
| | expression. |
| Bavachin | Weak antioxidant. |
| bis(7)-Tacrine (1,7-N-heptylene-bis-9,9′-amino- | Protects against hydrogen peroxide induced apoptosis |
| 1,2,3,4-tetrahydro-acridine) (CAS 224445-12-9) | |
| Caffeic acid | Antioxidant |
| Caffeic acid methyl ester | Antioxidant |
| Caffeic acid n-octyl ester | Antioxidant, Suppressor of inducible nitric oxide synthase |
| | (iNOS; NOS II). |
| Carazostatin | Free radical scavenger |
| Carnosic acid | Antioxidant |
| Carnosine | Antioxidant. |
| Catechin | Antioxidant flavonoid. Free radical scavenger. |
| Celastrol | Antioxidant. |
| Chlorogenic acid | Antioxidant. |
| Chrysin | Antioxidant flavonoid. |
| Curcumin | Antioxidant. |
| Cyanidin chloride | Antioxidant flavonoid. Nitric oxide (NO) scavenger. |
| Cyclosporin H | Inhibits formyl peptide-induced superoxide formation |
| CYPMPO | Free radical spin trap with excellent trapping capabilities |
| | toward hydroxyl and superoxide radicals |
| Daphnetin | Antioxidant. |
| Delphinidin chloride | Antioxidant. |
| Dihydrocapsaicin | Antioxidant |
| Diosmin | Inhibits lipopolysaccharide (LPS)-induced endothelial |
| | cytotoxicity. |
| DL-α-Lipoic acid | Antioxidant |
| Ellagic acid•dihydrate | Polyphenol antioxidant |
| Ebselen | Peroxynitrite (ONOO⁻) scavenger. |
| Epigallocatechin gallate | Antioxidant |
| Esculin•hydrate | Antioxidant used as a skin protectant. Reduces ROS levels. |
| Ethyl pyruvate | Inhibitor of ROS-mediated toxicity (peroxide scavenger) |
| Eugenol | Antioxidant |
| Formononetin | Inhibits lecithin peroxidation induced by hydroxyl radicals. |
| Gallotannin | Cytoprotective in oxidatively stressed cells. Inhibitor of |
| | endothelial nitric oxide synthase (eNOS; NOS III) and weak |
| | inhibitor of inducible (iNOS; NOS II) and neuronal nitric |
| | oxide synthase (nNOS; NOS I). |
| Gliotoxin | Antioxidant |
| Hesperetin | Antioxidant flavonoid. |
| Isorhamnetin | Antioxidant |
| Kaempferol | Antioxidant flavonoid. |
| L-(+)-Ascorbic acid | Antioxidant |
| Malvidin chloride | Antioxidant flavonoid. |
| Mannitol | Quenches ROS (hydroxyl radicals) |
| MnTBAP chloride (Manganese (III) tetrakis (4- | Potent inhibitor of peroxynitrite-induced oxidative reactions |
| benzoic acid)porphyrin chloride) | |
| MnTMPyP•pentachloride (Manganese (III) | Catalyzes the dismutation of O₂⁻ even in the presence of |
| tetrakis (1-methyl-4-pyridyl)porphyrin) | excess EDTA. |
| Morin | Antioxidant flavonoid. |
| Myricetin | Antioxidant flavonoid. |
| N-Acetyl-L-cysteine | Free radical scavenger (general) |
| Naringenin | Antioxidant flavonoid. |
| Pelargonidin chloride | Antioxidant flavonoid. Nitric oxide scavenger. |
| Peonidin chloride | Antioxidant flavonoid. |
| Psoralidin | Shows strong antioxidant activity |
| Pyrrolostatin | Potent inhibitor of lipid peroxidation, Free radical scavenger. |
| Pyruvate | Acts as an NADH trap and ROS scavenger (specifically, |
| | peroxydes) |
| Quercetin•dihydrate | Antioxidant flavonoid. Inhibits the production of nitric oxide |
| | (NO). Inhibits myeloperoxidase (HOCl). |
| Resveratrol | Inhibits the hydroperoxidase activity of COX-1. Antioxidant. |
| | Protects against 4-hydroxynonenal (4-HNE) induced |
| | oxidative stress and apoptosis. |
| Rosmarinic acid | Antioxidant. Inhibitor of lipid peroxidation, |
| Sauchinone | Inhibitor of LPS-inducible iNOS (NOS II), reducing ROS |
| | generation. |
| Silybin | Blocks the production of superoxide in Kupffer cells. |
| | Antioxidant. Free radical scavenger. |
| Sulfinpyrazone | Has free radical scavenging properties. |
| Taurine | HOCl scavenger |
| Taxifolin | Antioxidant flavonoid. |
| TEMPOL | Free radical scavenger useful for both in vivo and in vitro |
| | experiments. |
| Tiliroside | Free radical scavenger. Inhibits the production of the |
| | inflammatory mediators nitric oxide (NO), TNF-α and IL-12 |
| | in activated macrophages. |
| Tocopherol (α, β, δ, and γ) | Forms of vitamin E, known for antioxidant activity |
| | Suppression of nitric oxide toxicity |
| Tocotrienols (α, β, δ, and γ) | Forms of vitamin E, known for antioxidant activity |
| Trihydroxyethylrutin | Free radical scavenger. Antioxidant |
| tris(Dicarboxymethylene)fullerene-C3 | Water soluble neuroprotective antioxidant, both in vitro and |
| | in vivo |
| Tiron | Superoxide scavenger |
| U-74389G (21-(4-(2,6-di-1-Pyrrolidinyl-4- | Lazaroid inhibitor of iron-dependent lipid peroxidation. |
| pyrimidinyl)-1-piperazinyl)-pregna-1,4,9(11)- | Antioxidant. |
| triene-3,20-dione•(Z)-2-butenedioate) | |
| β-Carotene | Antioxidant. |
| Cinnamtannin B-1 | Potent antioxidant |
| EUK 118 (CAS 186299-34-3) | Catalytic scavenger of reactive oxygen species |
| EUK 124 (CAS 186299-35-4) | Catalytic scavenger of reactive oxygen species |
| Free radical donors/generators: | |
| 3-Carboxy-2,2,5,5-tetramethyl-1-pyrrolidine-1- | Free radical compound. |
| oxyl, free radical | |
| AAPH (CAS 2997-92-4) | Water-soluble azo compound which is used as a free radical |
| | generator in the study of lipid peroxidation and the |
| | characterization of antioxidants. |
| AMVN (CAS 4419-11-8) | A synthetic azo compound that dissociates spontaneously to |
| | form carbon-centered free radicals. |
| Antimycin A | ROS generator |
| EUK 134 (CAS 81065-76-1) | Synthetic manganese-porphyrin complex that acts as |
| | scavenger for oxidative species such as peroxynitrite, |
| | superoxide, and hydrogen peroxide. |
| Galvinoxyl, free radical | Free radical compound. |
| Hesperetin (CAS 520-33-2) | Antioxidant flavinoid |
| Hydrogen peroxide | Free radical generator |
| Menadione | Free radical generator |
| PMA (phorbol myristate acetate) | Free radical generator |
| PTMIO, free radical | Free radical similar to 4-Amino-TEMPO |
| Pyocyanin | Undergoes nonenzymatic reduction by NADPH, which |
| | produces hydrogen peroxide and depletes intracellular |
| | glutathione levels, causing oxidative stress in susceptible |
| | cells. |
| Pyrogallol | Induces superoxide production in the live cells |
| SOTS-1 (Di-(4-Carboxybenzyl)Hyponitrite) | An azo-compound that can be thermally decomposed in |
| | aqueous solution to generate superoxide radical anion at a |
| | constant, controlled rate. |
| TBHP (tert-butyl hydroperoxide) | Free radical generator |
| Trans-4,5-epoxy-2(E)-Decenal (3-(3- | Elucidates the effects of peroxidative damage. |
| pentyloxiranyl)-2E-propenal) (CAS 134454-31- | |
| 2) | |
| Xylitol | NADH-generating compound that enhances ROS production |

Again, due to the relative infancy of the RHS field, selective activators and inhibitors are generally lacking for these reactive species. However, glutathione (GSH), is the prime in vivo scavenger for HOCl. N-acetyl-L-cysteine, desferrioxamine and uric acid will also scavenge HOCl. Taurine is considered a relatively selective scavenger of HOCl. In the presence of ammonia HOBr is scavenged in a fast reaction forming bromamine (NH₂Br) and dibromamine (NHBr₂), which are not believed to be oxidized to bromate directly. Nitrite can be used as a scavenger for HOCl and ClO₂. Enzyme inhibitors of myeloperoxidase can also be considered as inhibitors of RHS. Flavonoids are known to act as antioxidative and anti-inflammatory agents. For example, quercetin is an example of a flavinoid myeloperoxidase inhibitor that in turn inhibits HOCl production. US 20050234036 describes thioxanthine derivatives as myeloperoxidase inhibitors. Azide, cyanide, naphthalenes and methimazole are also considered inhibitors of myeleoperoxidase activity.

### Dye/Inhibitor Combinations

Table 5 below provides yet further information on the possible combination of dyes and inhibitors one can use to detect a particular ROS/RNS type. In Table 5, the sample should be stained with three dyes (in this case, DAQ, DCFDA and HE). The presence of the signal in the appropriate spectral region (green, orange or red fluorescence) will indicate the presence of certain ROS/RNS (listed in the appropriate columns of the Table 5). For example, having green and red signal will indicate the presence of NO and one or more of the following types of species—peroxides, hydroxyl radicals, or peroxynitrite.

To further profile ROS/RNS, parallel samples may be pretreated with inhibitors. The presence of the signal in one of the spectral regions will indicate certain ROS/RNS type (listed in the appropriate columns of Table 5). For example, treatment with cPTIO (NO scavenger and non-specific nitric oxide synthase inhibitor) will eliminate red signal (NO). One still will be able to see, however, orange signal indicating superoxide presence. It should be appreciated that more than one inhibitor can be used. For example, if upon pretreatment with ebselen, one detected a significant decrease in green signal, it is a strong indication of peroxynitrite presence. Remaining green signal can be induced with peroxides and/or hydroxyl radicals; therefore, the next step will be the treatment of the sample with mannitol (inhibitor of hydroxyl radicals) or pyruvate (peroxide scavenger) to indicate or eliminate the presence of corresponding species.

TABLE 5
Various combinations of dyes and inhibitors (see explanation above).

| | DCFDA | HE | DAQ |
|---|---|---|---|
| No | R-OOH | O₂⁻ | NO |
| Inhibitors | OH⁻ | | |
| | ONOO⁻ | | |
| cPTIO | R-OOH | O₂⁻ | No signal |
| | OH⁻ | | |
| | (ONOO⁻) | | |
| NAC | No signal | No signal | NO |
| Tiron | R-OOH | No signal | NO |
| | OH⁻ | | |
| | (ONOO⁻) | | |
| Ebselen | R-OOH | (O₂⁻) | (NO) |
| | OH⁻ | | |
| Pyruvate | OH⁻ | O₂⁻ | NO |
| | ONOO⁻ | | |
| Mannitol | R-OOH | O₂⁻ | NO |
| | ONOO⁻ | | |
| cPTIO & | R-OOH | O2⁻ | No signal |
| mannitol | (ONOO−) | | |
| cPTIO & | OH⁻ | O2⁻ | No signal |
| pyruvate | (ONOO−) | | |
| Ebselen & | R-OOH | (O2⁻) | (NO) |
| Mannitol | | | |
| Pyruvate | OH⁻ | (O2⁻) | (NO) |
| & Ebselen | | | |
| Mannitol & | ONOO− | O2⁻ | NO |
| Pyruvate | | | |
| Mannitol | ONOO− | O2⁻ | No signal |
| cPTIO | | | |
| Pyruvate | | | |
| Mannitol | ONOO− | No signal | NO |
| Tiron | | | |
| Pyruvate | | | |

### ADDITIONAL EXAMPLES

The next two tables (Tables 6 & 7) represent yet further examples to demonstrate how the above information in Table 5 can be applied to profile or monitor ROS/RNS species in living cells, (as well as tissues, organs or organisms and subcellular organelles).

In the example shown below in Table 6, a solution containing all three probes are added to each sample, followed by addition of appropriate inhibitors.

In the example (Table 6), Sample A provides different information depending upon the particular wavelength being monitored. With Filter #1, the presence and location of R—OOH, OH⁻ and ONOO⁻ are simultaneously evaluated, whereas O₂⁻ and NO are seen with Filter #2 and Filter #3, respectively. In many cases, it may be desirable to evaluate R—OOH, OH⁻ and ONOO⁻ separately as opposed to collectively as in Sample A. As such, Sample B will allow evaluation of OH⁻ separately from R—OOH and ONOO⁻ seen with Sample A and that example, while in the last example, Sample C will evaluate ONOO⁻ separately while also allowing a reconfirming of O₂₋ and NO with Filter #2 and Filter #3.

The presence of R—OOH alone may also be indirectly evaluated by a comparison of Sample A with Sample B and Sample C

TABLE 6
Examples of ROS/RNS profiling

| | | | Filter #1 | Filter #2 | Filter #3 |
|---|---|---|---|---|---|
| | | Inhibitor | (Species | (Species | (Species |
| Example | Probe | Added | Detected) | Detected) | Detected) |
| Sample A | DCFDA | None | R—OOH | O₂⁻ | NO |
| | HE | | OH⁻ | | |
| | DAQ | | ONOO⁻ | | |
| Sample B | DCFDA | Pyruvate/ | OH⁻ | (O₂⁻) | (NO) |
| | HE | Ebselen | | | |
| | DAQ | | | | |
| Sample C | DCFDA | Mannitol/ | ONOO⁻ | O₂⁻ | NO |
| | HE | Pyruvate | | | |
| | DAQ | | | | |

The example below in Table 7 is similar to the setup in Table 6 above except that inhibitors would be added to each of the three samples. Thus, in Sample C, each of the filters allows evaluation of a single species (ONOO⁻, O₂⁻ and NO) while R—OOH and OH⁻ are individually evaluated in Sample A and Sample B.

TABLE 7
Examples of ROS/RNS species profiling

| | | | Filter | Filter | Filter |
|---|---|---|---|---|---|
| | | | #1 | #2 | #3 |
| | | Inhibitor | (Species | (Species | (Species |
| Example | Probe | Added | Detected) | Detected) | Detected) |
| Sample A | DCFDA | Ebselen/ | R—OOH | (O₂⁻) | (NO) |
| | HE | Mannitol | | | |
| | DAQ | | | | |
| Sample B | DCFDA | Ebselen/ | OH⁻ | (O₂⁻) | (NO) |
| | HE | Pyruvate | | | |
| | DAQ | | | | |
| Sample C | DCFDA | Mannitol/ | ONOO⁻ | O₂⁻ | NO |
| | HE | Pyruvate | | | |
| | DAQ | | | | |

The following two tables (Tables 8 & 9) represent variations in the methods shown in Table 6 and Table 7 above.

It should be noted that although three probes are present in one sample (Sample A), the HE and NO probes are not required to be present in the samples that are only intended to generate information on OH⁻ and ONOO⁻ (Sample B and Sample C). As such, a reagent solution can be made with appropriate Probe/Inhibitor already combined together and the various combinations can be applied to each of the samples. Thus, Sample A has all three probes since readings are taken at each wavelength while Sample B and Sample C only have the probe that will be read with Filter #1.

TABLE 8
Examples of ROS/RNS profiling

| | | | Filter | Filter | Filter |
|---|---|---|---|---|---|
| | | | #1 | #2 | #3 |
| | | Inhibitor | (Species | (Species | (Species |
| Example | Probe | Added | Detected) | Detected) | Detected) |
| Sample A | DCFDA | None | R—OOH | O₂⁻ | NO |
| | HE | | OH⁻ | | |
| | NO | | ONOO⁻ | | |
| Sample B | DCFDA | Pyruvate/ | OH⁻ | — | — |
| | | Ebselen | | | |
| Sample C | DCFDA | Mannitol/ | ONOO⁻ | — | — |
| | | Pyruvate | | | |

In a similar fashion, the combinations previously shown in Table 7 can be made with each probe/Inhibitor mixture as a single reagent that is subsequently applied to Sample A, Sample B and Sample C. In this way, a read-out will be obtained for ONOO⁻, O₂⁻ and NO with each wavelength in Sample C and R—OOH and OH⁻ being evaluated with Filter #1 only (and DCFDA only) for Sample A and Sample B, respectively.

TABLE 9
Examples of ROS/RNS species profiling

| | | | Filter | Filter | Filter |
|---|---|---|---|---|---|
| | | | #1 | #2 | #3 |
| | | Inhibitor | (Species | (Species | (Species |
| Example | Probe | Added | Detected) | Detected) | Detected) |
| Sample A | DCFDA | Ebselen/ | R—OOH | — | — |
| | | Mannitol | | | |
| Sample B | DCFDA | Ebselen/ | OH⁻ | — | — |
| | | Pyruvate | | | |
| Sample C | DCFDA | Mannitol/ | ONOO⁻ | O₂⁻ | NO |
| | HE | Pyruvate | | | |
| | NO | | | | |

Set forth below in Table 10 are additional sets of probes which can be employed to detect ROS, RNS and RHS species, and their combinations. Excitation and emission characteristics and the selected reactive species are provided in Table 10 below.

TABLE 10
Probes & Their Characteristics For ROS, RNS & RHS

| Probe | Excitation (nm) | Emission (nm) | Selectivity |
|---|---|---|---|
| Set 1 | | | |
| SNAPF | 625 | 735 | Hypochlorite |
| DAQ | 488 | 580 | Nitric oxide |
| HE | 500 | 530 | Superoxide |
| Set 2 | | | |
| HKOCI-1 | 520 | 541 | Hypochlorite |
| APF | 488 | 515 | ⁻OH, ONOO⁻, HOCl⁻ |
| Terephtalic | 326 | 432 | Hydroxyl radical |
| acid | | | |
| Set 3 | | | |
| HySOx | 552 | 576 | Hypochlorite |
| DAF-2 | 495 | 515 | RNS |
| DHR | 500 | 536 | ROS |
| Set 4 | | | |
| APF | 488 | 515 | ⁻OH, ONOO⁻, HOCl⁻ |
| NFDS-1 | 602 | 662 | Hydrogen peroxide |
| DPBF | 410 | 455 | superoxide |

The methods of the present invention developed from the observations described above and from the experimental work provided below in the Preferred Embodiment section. One such method is useful for profiling the status of reactive oxygen species (ROS) and reactive nitrogen species (RNS) in living cells or subcellular organelles, or both living cells and subcellular organelles. Briefly, this method comprises providing (A) (i) at least one sample of the living cells and/or cellular organelles to be profiled for ROS/RNS; and (ii) three or more indicator probes capable of providing signals.

The living cells may be contained in tissue, an organ or an organism. The subcellular organelles include a great many examples such as mitochondria, peroxisomes, cytosol, vesicles, lysosomes, plasma membranes, chloroplasts, nuclei, nucleoli, inner mitochondrial matrices, inner mitochondrial membranes, intermembrane spaces, outer mitochondrial membranes, secretory vesicles, endoplasmic reticuli, golgi bodies, phagosomes, endosomes, exosomes, plasma membranes, microtubules, microfilaments, intermediate filaments, filopodia, ruffles, lamellipodia, sarcomeres, focal contacts, podosomes, ribosomes, microsomes, lipid rafts, nuclear membranes, chloroplasts and cell walls, or a combination of any of the foregoing. Mitochondria and peroxisomes are especially preferred as subcellular organelles. The subcellular organelles may be contained in a cell extract or in cells themselves.

The indicator probes are independently selected from (a) global reactive species probes for detecting or quantifying in living cells or subcellular organelles oxidative stress, nitrative stress, or halogenating stress (and combinations thereof); and (b) selective reactive species probes for detecting specific ROS species, specific RNS species, or both. The sample containing living cells and/or subcellular organelles is initially contacted (B) with the three or more indicator probes to generate signals; and these signals are measured (C), thereby providing a status profile of specific ROS/RNS species in the living cells and/or subcellular organelles.

Reactive species for profiling have been described or listed above. For the sake of completeness, reactive oxygen species (ROS) are selected from superoxide (O₂^{•-}), hydroperoxy (HO^{•}₂), hydrogen peroxide (H₂O₂), peroxynitrite (ONOO⁻), hypochlorous acid (⁻OHCl), hypobromous acid (⁻OHBr), hydroxyl radical (HO^{•}), peroxy radical (ROO^{•}), alkoxy radical (RO^{•}), singlet oxygen (¹O₂), lipid peroxides, lipid peroxyradicals, and lipid alkoxyl radicals, or a combination of any of the foregoing. Among reactive nitrogen species (RNS) to be profiled are those selected from nitric oxide (NO), nitrogen dioxide radical (^{•}NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) and dinitrogen trioxide (N₂O₃), or a combination of any of the foregoing. Among reactive halogen species (RHS) to be profiled are those selected from hypochlorous acid (HOCl), hypochlorite ion (ClO⁻) monochloramine (NH₂Cl), chlorine dioxide (ClO₂), nitryl chloride (NO₂Cl), chlorine (Cl₂), bromine (Br₂), bromochloride (BrCl), hypobromous acid (HOBr), hypobromite ion (BrO⁻) and all three bromamine species (NH₂Br, NHBr₂, NBr₃), or a combination of any of the foregoing. The just-described lists of reactive oxygen species, reactive halogen species and reactive nitrogen species are not intended to be limiting.

As indicated above, the three or more indicator probes can take the form of so-called global reactive species probes or selective reactive species, and these can be in various combinations. For example, one could use three or more global reactive species probes, or three or more selective reactive species probes. Or, one could use two or more global probes and one selective reactive species probe. Alternatively, one could use two or more selective reactive species probes and a single global reactive species probe. In a preferred aspect of the present invention, the indicator probes are fluorescent and generate fluorescent signals.

In certain embodiments, the global reactive species probes can comprise but are not limited to DCFDA, dihydrorhodamine 123 (DHR), C₁₁-BODIPY, DAF-2, DAR-4M, dihydrocalcein and a Redox-sensitive Green Fluorescent Protein (roGFP), or a combination of any of the foregoing. Among selective reactive species probes are those comprising any of 2-(2-pyridyl)-benzothiazoline, Amplex Red, APF, Bis-2,4-dinitrobenzenesulfonyl fluoressceins, BODIPY FL EDA, CCA/SECCA, copper (II) fluorescein, CsPA (cis-parinaric acid), DAC (diaminocyanine), DAMBO-P^{H}, DAQ, DHE, DMA, DMAX, Dobz derivatives, DPAX (9-[2-(3-carboxyl-9,10-diphenyl)anthryl]-6-hydroxy-3H-xanthen-3-one), DPBF (1,3-diphenylisobenzofuran), DPPEA-HC, DPPEC, DPPP (diphenyl-1-pyrenylphosphine), FL₅, HKOCl-1, homovanilic acid, HPF, HySOX, metal-based turn-on fluoresecent probes, MitoPY1, Mito-SOX, MitoTracker Orange (dihydrotetramethyl-rosamine), NBD-Cl (4-chloro-7-nitrobenzo-2-oxa-1,3-diazole), NFDS-1, pentafluorobenzene-sulfonyl fluorescein, Peroxifluor-1, Peroxycrimson-1, Peroxygreen-1, Peroxyresorufin-1, o-Phenylenediamine derivatives, scopoletin, Spy-HP, Rhodamine spirolactam, SNAPF, Singlet Oxygen Sensor Green, Terephtalic acid and TMDA BODIPY, a selective Redox-sensitive Green Fluorescent Protein (roGFP) and HyPer, or a combination of any of the foregoing. Again, the foregoing list of selective probes is not intended to limit or constrain the practitioner in his or her choice of probe candidates.

Other useful components can also be employed with the present invention and method. These other useful components include (ii) (c) one or more inhibitors or scavengers of reactive species generation selected from ROS and/or RNS, and/or (ii) (d) one or more activators, donors or generators of reactive species generation selected from ROS and/or RNS. Thus, a combination of such inhibitors/scavengers and activators/donors/generators can be usefully employed in these methods. Briefly, the contacting step (B) can be carried out by contacting the living cells and/or subcellular organelles with the three or more indicator probes and either with the one or more inhibitors or scavengers (ii) (c), the one or more activators, donors or generators (ii) (d), or a combination of inhibitors/scavengers and activators/donors/generators.

Thus, the profiling method of the present invention can likewise comprise the step of (A) providing: (i) at least one sample of living cells and/or cellular organelles for ROS/RNS profiling; (ii) three or more indicator probes independently selected from (a) global reactive species probes for detecting or quantifying in living cells and/or subcellular organelles oxidative stress, nitrative stress, or halogenating stress (and combinations thereof); (b) selective reactive species probes for detecting ROS species and/or RNS species; (iii) (a) one or more inhibitors or scavengers of reactive species generation selected from ROS and/or RNS; and optionally, (b) one or more activators, donors or generators of reactive species generation selected from ROS and/or RNS. The sample of living cells and/or subcellular organelles is contacted (B) with the three or more indicator probes to generate signals which are measured (C), thereby providing a status profile of specific ROS/RNS species in the sample of living cells and/or subcellular organelles.

There are diverse manners by which the various components of the profiling method can vary and take different forms. For example, the living cells and/or subcellular organelles can be simultaneously contacted with the three or more indicator probes and the one or more inhibitors/scavengers and/or the one or more activators/donors/generators. Alternatively, the living cells and/or subcellular organelles can be contacted with the three or more indicator probes before contacting the living cells and/or subcellular organelles with the inhibitors/scavengers, and/or the activators/donors/generators. Or, the living cells and/or subcellular organelles can be contacted with the three or more indicator probes after contacting the living cells and/or subcellular organelles with the inhibitors/scavengers and/or theactivators/donors/generators.

The inhibitors and scavengers have been described above, but for the sake of completeness, these can comprise any of N-acteyl cysteine, 7-nitroindazole, cPTIO, L-NAME, L-NMNA and L-NNA, and free-radical scavengers, or a combination of any of the foregoing, just to name a few of the preferred candidates. Among free-radical scavengers and not intended to be limiting are ebselen, mannitol, N-acetyl cysteine, pyruvate, Tiron and EUK, or a combination of any of the foregoing. The one or more activators, donors or generators (ii) (d) can preferably comprise NONOate, GEA, L-arginine, NOC, SIN-1, SNAP, sodium nitroprusside and free-radical donors/generators, or a combination of any of the foregoing. Such free-radical donors/generators include illustratively any of Antimycin A, pyocyanin, pyrogallol, PMA and TBHP, or a combination of any of the foregoing.

It should be pointed out that the profiling method of the present invention can be performed with two or more samples of living cells and/or subcellular organelles. Furthermore, the profiling method can be carried out with parallel samples.

Those skilled in the art will also appreciate that monitoring of such reactive species in living cells and/or subcellular organelles can be readily performed by carrying out a series of profiling methods. Successive profiling methods could be carried out in order to provide a means for monitoring over any period of time the physiological or pathophysiological processes of the organism from which the living cells and/or subcellular organelles have been obtained or isolated.

Also provided by the present invention is a method of quantifying signals from cells, organelles, cell regions and/or domains of cells of interest, or a combination of any of the foregoing. This quantification method comprises the steps of (A) providing: (i) a sample containing said cells of interest; (ii) at least one solution comprising: (I) three or more indicator probes independently selected from: (a) global probes for detecting or quantifying in living cells and/or subcellular organelles oxidative stress and/or nitrative stress and/or halogenating stress; (b) selective reactive species probes for detecting specific ROS species and/or specific RNS species; (II) one or more inhibitors of reactive species generation selected from ROS and/or RNS; and optionally, (III) one or more activators of reactive species generation selected from ROS and/or RNS. The cells of interest (i) are incubated (B) in the solution (ii) to generate signals from cells, organelles, cell regions or domains of said cells of interest or any of the foregoing. The generated signals are quantified (C).

It should be appreciated by those skilled in the art that the quantifying step (C) is conventionally carried out by several different means. These include any or all of the following:

comparing a normal state of said cells of interest to a perturbed state;

comparing unknown experimental samples to positive and/or negative control samples from said cells of interest;

comparing the ratio of signal strengths among different samples of said cells of interest; and

comparing unknown experimental samples of said cells of interest to calibration standards. The latter calibration standards can comprise microspheres or bead standards, or both.

It should also be appreciated that the quantifying step (c) can be conventionally carried out by counting, examining, and/or sorting suspensions of cells and/or subcellular organelles in a stream of fluid through an optical and/or electronic detection apparatus, e.g., a flow cytometer. The quantifying step (c) can also be carried out either by a direct means or after performing fractionation, extraction or liquification of the sample.

The generated signal is preferably fluorescent and the quantifying step (C) is preferably carried out by several different means. Such means can take the form of 1) an excitation source, 2) wavelength filters or diffraction gratings to isolate emission photons from excitation photons, or 3) a detector that registers emission photons and produces a recordable output. The recordable output can comprise an electrical signal or a photographic image, or both. All such means are known in the art and are available from a number of commercial sources.

When fluorescent signals are employed in this quantifying method, these signals are detected by a number of different means or instruments. These include any and all of the following: a fluorescence microscope, a flow cytometer, a confocal microscope, a fluorometer, a microplate reader, a high-content cell analysis system, a high-content cell screening system, cell microarray system (positional and/or nonpositional), a laser-scanning cytometer, a capillary electrophoresis apparatus or a microfluidic device, and a combination of any of the foregoing.

### Reagent Kits and Systems:

Commercial kits and systems are valuable because they eliminate the need for individual laboratories to optimize procedures, saving both time and resources. Commercial kits also allow better cross-comparison of results generated from different laboratories. The present invention additionally provides reagent kits, i.e., reagent combinations or means, comprising all of the essential elements required to conduct a desired assay method. The reagent system is presented in a commercially packaged form, as a composition or admixture where the compatibility of the reagents will allow, in a test kit, i.e., a packaged combination of one or more containers, devices or the like holding the necessary reagents, and usually written instructions for the performance of the assays. Reagent systems of the present invention include all configurations and compositions for performing the various labeling and staining formats described herein.

The reagent system will contain three or more fluorogenic indicators, generally comprising:
(1) one or more fluorogenic global ROS or RNS indicator;
(2) one or more fluorogenic indicator with selectivity for some sub-class of ROS or RNS analyte;
(3) optionally, one or more activators and/or inhibitors of ROS and/or RNS generation; and
(4) Instructions for usage of the included reagents.

More particularly, the present invention provides a kit for profiling the status of reactive oxygen species (ROS) and/or reactive nitrogen species (RNS) in living cells and/or subcellular organelles. In packaged combination, the kit comprises: (i) three or more indicator probes independently selected from: (a) global reactive species probes for detecting or quantifying in living cells and/or subcellular organelles oxidative stress, and/or nitrative stress and/or halogenating stress; and (b) selective reactive species probes for detecting specific ROS species and/or specific RNS species; (ii) buffers; and (iii) instructions therefore.

The reactive oxygen species (ROS), reactive nitrogen species (RNS), the global reactive species probes, the oxidative stress detection reagents, the selective reactive species probes, inducers, scavengers, activators, donors, generators, free-radical scavengers and free-radical donors/generators have all been described above previously and need not require further elaboration with respect to the present kit.

Generic instruction, as well as specific instructions for the use of the reagents on particular instruments, such as a wide-field microscope, confocal microscope, flow cytometer or microplate-based detection platform may be provided. Recommendations regarding filter sets and/or illumination sources for optimal performance of the reagents for a particular application also may be provided.

A test kit form designed to directly monitor real time ROS/RNS production in live cells, for example, can contain an indicator of global ROS generation (e.g. DCFH), an indicator of superoxide generation (e.g. HE), an indicator of nitric oxide generation (e.g. DAQ) and additional ancillary chemicals, such as dilution buffer (e.g. phosphate-buffered saline), NO generating compound (e.g. N-(acetoxy)-3-nitrosothiovaline (SNAP) or L-arginine), general ROS generating compound (e.g. pyocyanin), NO scavenging compound (e.g. 2-(4-carboxyphenyl)-4,5-dihydro-4,4,5,5-tetramethyl-1H-imidazolyl-1-oxy-3-oxide, potassium salt (c-PTIO)), and general ROS scavenging compound (e.g. N-acetyl-L-cysteine). In some instances one or more fluorogenic compound may be combined within a single container for easier use.

The present invention also provides a novel system for profiling or monitoring the status of reactive oxygen species (ROS) and/or reactive nitrogen species (RNS) in living cells and/or subcellular organelles. This system comprises (i) container means for three or more indicator probes independently selected from (a) global reactive species probes for detecting or quantifying oxidative stress and/or nitrative stress or halogenating stress (and combinations thereof) in living cells and/or subcellular organelles; and (b) selective reactive species probes for detecting specific ROS species and/or RNS species; (ii) other container means for providing optional reagents or components comprising: (c) one or more inhibitors or scavengers of reactive species generation selected from ROS and/or RNS; and (d) one or more activators, donors or generators of reactive species generation selected from ROS and/or RNS; (iii) an instrument, a device or means for introducing the probes and the optional reagents or components to a sample of living cells or subcellular organelles; and (iv) measuring means to measure signal generation. The measuring means can take the form of instruments or devices including a fluorescence microscope, a flow cytometer, a confocal microscope, a fluorometer, a microplate reader, a high-content cell analysis system, a high-content cell screening system, cell microarray system (positional and/or nonpositional), a laser-scanning cytometer, a capillary electrophoresis apparatus or a microfluidic device, and a combination of any of the foregoing.

All of the components named in the novel system have already been described above and require no specific elaboration with respect to their identity or their use in this system.

### Diagnostic and Prognostic Application:

A number of diseases are associated with excessive ROS generation, produced mostly in the mitochondria as byproducts of cell respiration or alternatively resulting from neutrophil activation. Generally speaking, in a plethora of diseases the redox state of cellular systems becomes persistently shifted toward oxidation, resulting in a sequence of pathophysiological events. Aberrant ROS profiles are a hallmark of mitochondrial-associated diseases, such as various mitochondrial encephalomyopathies, including myoclonic epilepsy associated with ragged-red fibers (MERRF). Additionally, a range of other diseases may manifest themselves thru altered ROS/RHS/RNS production, including sepsis, cataract formation, rheumatoid arthritis, diabetes mellitus, Parkinson's disease and Alzheimer's disease. Additionally, hyperthyroidism can cause elevation in hormone secretion, leading to perturbations in overall metabolic status. The altered state causes increased generation of ROS, leading to oxidative stress in these patients. Also, Chlamydia pneumoniae infection induces nitric oxide synthase and lipoxygenase-dependent production of ROS/RNS in platelets. Furthermore, Chronic Granulomatous Disease (CGD) is an inherited disorder characterized by defective killing of microorganisms due to genetic mutations in components of the NADPH oxidase system, thus altering ROS profiles in granulocytes. Finally, exposure to environmental toxins, such as heavy metals, polycyclic aromatic hydrocarbons and pesticides, as well as exposure to chemotherapeutic drugs or radiation can alter ROS/RNS profiles.

Flow cytometric techniques have previously been developed for quantifying oxidative burst activity at the single cell level using fluorescent probes such as DCFH or dihydrorhodamine. The specific form of ROS being measured using this method is not, however, clearly defined. The present invention has applications in rapid flow cytometry-based or HCS/HCA-based diagnosis of certain diseases using whole-blood or isolated blood cell types, such as neutrophils, eosinophils, monocytes or platelets, providing unprecedented ability to categorize the types and quantities of ROS/RNS associated with the condition being examined. The present invention is also readily applied to other naturally suspended individual cells of human or animal origin, as well as readily accessible cells that may require disaggregation into single cells in suspension before analysis. This ROS/RNS fingerprinting strategy should permit better diagnosis of disease thru better characterization of the reactive species generated. The multi-parametric analysis of ROS/RNS using fluorescent probes is more economical than alternate methods based upon antibody conjugates. While the ROS/RNS indicators may be used in conjunction with antibody-based detection modalities, their use in the absence of antibody-based probes allows analysis without additional sample preparation steps, such as cell fixation and permeabilization. The ROS/RNS fingerprinting technology would also be useful in assessing the success of therapeutic interventions, such as implementation of gene therapy technologies for correction of inherited disorders such as CGD.

The examples which follow are set forth to illustrate various aspects of the present invention but are not intended in any way to limit its scope as more particularly set forth and defined in the claims that follow thereafter.

### DESCRIPTION OF THE PREFERRED EMBODIMENTS

### Example 1

### Detection of ROS/RNS Production in HeLa Cells by Wide-Field Fluorescence Microscopy Using a Triple-Staining Protocol

Human cervical adenocarcinoma epithelial cell line HeLa was obtained from ATCC (ATTC, Manassas, Va.) and was routinely cultured in Dulbecco's modified eagle medium with low glucose (Sigma-Aldrich, St. Louis, Mo.), supplemented with 10% fetal bovine serum heat inactivated (ATCC) and 100 U/ml penicillin, 100 μg/ml streptomycin (Sigma). Cell cultures were maintained in an incubator at 37° C., with 5% CO₂ atmosphere. Three ROS/RNS fluorescent probes were dissolved in anhydrous DMF at the following concentrations: DAQ-20 mM (a 400× stock solution), DCFDA-5 mM (a 5000× stock solutions), DHE-5 mM (a 5000× stock solution). Anhydrous organic solvents should be used with DMF being the first choice, since DMSO is a hydroxyl radical scavenger and its presence may affect ROS/RNS production in cellular systems. Stock solutions of the dyes were aliquoted and stored at −20° C. The day before the experiment, HeLa cells were seeded on multiwell microscope slides (Cel-Line™ Brand, Portsmouth, N.H.) at a density of 2×10⁴ cells per well. On the next day, the cells were loaded with 50 μM of DAQ, 1 μM of DCFDA and HE (all dilutions were made in growth medium) for 2 h, 37° C. Then the medium containing dyes was removed, the cells were briefly washed with PBS and induced with L-arginine (1 mM), pyocyanin (100 μM) or their combination for 20 min. Then the inducer-containing medium was removed, and after a brief wash with PBS, the cells were overlaid with a cover slip and observed under wide field fluorescence Olympus microscope equipped with the standard set of filters described in Table 11. To confirm specific detection of ROS/RNS, parallel samples of HeLa cells were pretreated for 1 h with 5 mM NAC (general ROS scavenger), or 20 μM cPTIO (general NO scavenger and non-specific NOS inhibitor). Pretreated cells were induced as described earlier, overlaid with a cover slip and observed under fluorescence microscope.

As demonstrated further below (see Table 11), each of these three probes (HPF, APF and DCFH) has a different reactivity profile when screened against a battery of ROS and RNS. It should be noted that the three dyes cited in Table 11 display essentially the same excitation/emission profiles. Thus, these three probes cannot be combined together to provide simultaneous readouts of different ROS. While hypochlorite can be selectively detected by monitoring the response of APF relative to HPF, this detection cannot be performed in the same well using the same cells. Similarly, insight regarding the generation of the alkylperoxyl radical cannot be obtained using combinations of two or three of these dyes, despite DCFH having almost two-orders of magnitude greater sensitivity to this analyte compared with HPF or APF.

The detection of RHS by fluorescent indicator dyes can be considered at present a discipline in its infancy. Intracellular HOCl can be monitored under certain circumstances using the global ROS fluorescent probes 2′,7′ dichlorodihydrofluorescein diacetate or the closely related 5-(and -6)-chloromethyl-2′,7′-dichlorodihydrofluorescein diacetate, acetyl ester (Pi et al Toxicology and Applied Pharmacology 226: 236-243 (2008)). As summarized in Table 11, however, APF is a vastly superior probe for this application. A rhodamine-based probe, HySOx, and a sulfonaphthoaminophenyl fluorescein-based probe, SNAPF, were recently described for the selective detection of HOCl (Kenmoku J. Am. Chem. Soc., 129, 7313-7318 (2007); Shepherd et al Chem. Biol., 14, 1221-1231 (2007)). A BODIPY dye-based fluorescent probe, HKOCl-1, has also been successfully developed for the detection of hypochlorous acid on the basis of a specific reaction with p-methoxyphenol (Sun et al Org. Lett., 10, 2171-2174 (2008)). Taurine, is another molecule often used to detect chlorination activity (Spalteholz et al Archives of Biochemistry and Biophysics 445: 225-234 (2006)). The resulting taurine chloramine formation, is used as an index of residual HOCl concentration and is monitored spectophotometrically. The bromine and chlorine species also react with ABTS (2,2-azino-bis(3-ethylbenzothiazoline)-6-sulfonic acid-diammonium salt) to form a green colored product that can be measured spectrophotometrically at 405 or 728 nm (Pinkernell et al Wat. Res. 34, 4343-4350 (2000)).

TABLE 11
Fluorescence increase of HPF, APF, and DCFH in various
ROS-generating systems.

| ROS | HPF | APF | DCFH |
|---|---|---|---|
| ⁻OH | 730 | 1200 | 7400 |
| ONOO⁻ | 120 | 560 | 6600 |
| ⁻OCl | 6 | 3600 | 86 |
| ¹O₂ | 5 | 9 | 26 |
| O₂⁻ | 8 | 6 | 67 |
| H₂O₂ | 2 | 1 | 190 |
| NO | 6 | 1 | 150 |
| ROO⁻ | 17 | 2 | 710 |

Source: Setsukinai et al., JBC, 2003: 278 (5), pp.3170-3175

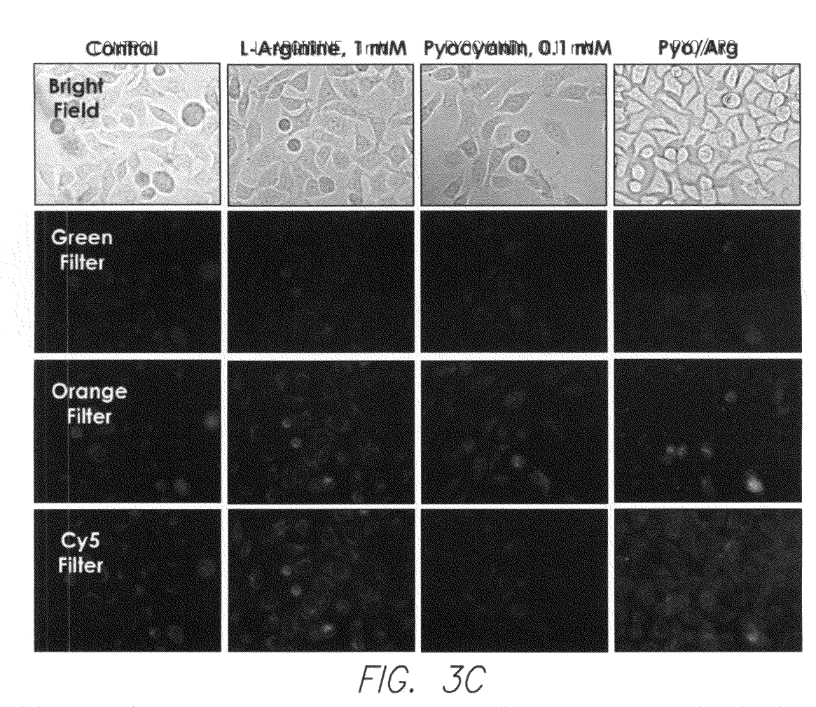

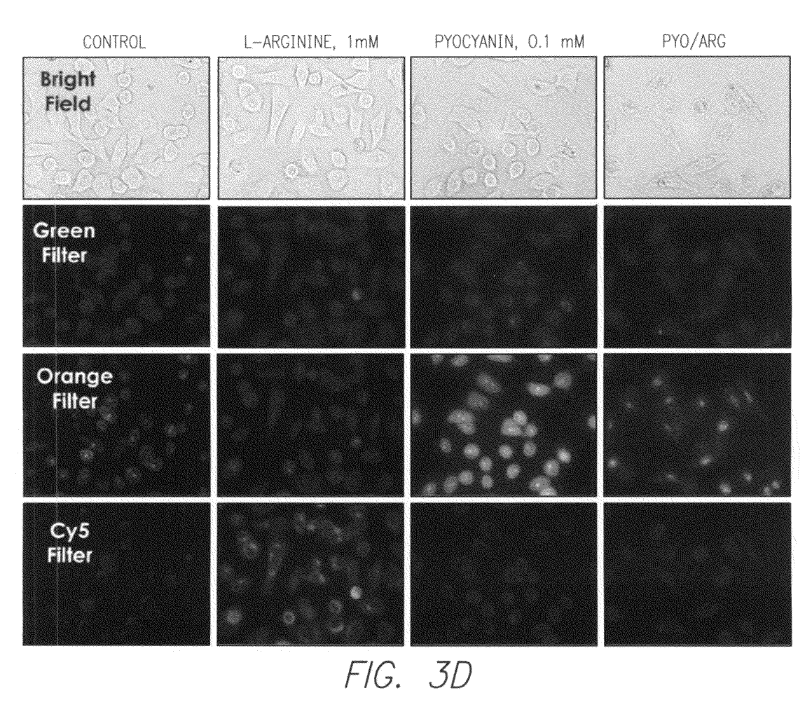

As shown on FIG. 3A, L-arginine treatment led to an extensive NO production that was detected by oxidized DAQ fluorescence using a Cy5 filter, while pyocyanin treatment did not affect NO production in HeLa cells. In turn, pyocyanin induced superoxide production (detected by HE fluorescence using an orange filter) and to a lesser extent, produced other types of ROS (detected by DCF fluorescence using a green filter). Combinations of these two reagents (L-arginine and pyocyanin) led, however, to a change in the ROS/RNS profile: there was less NO and superoxide detected after combination treatment, and significant increase in green staining was observed. NO and superoxide reacted in the system to yield peroxynitrite that was efficiently detected by DCF staining. Further confirmation of the observed results was obtained by using the specific ROS/RNS inhibitors: cPTIO (NO scavenger and general NOS inhibitor, FIG. 3B), NAC (general ROS inhibitor, FIG. 3C) or ebselen (specific peroxynitrite scavenger, FIG. 3D). cPTIO (20 μM) pretreatment completely abrogated NO production upon L-arginine induction but did not affect ROS production induced by pyocyanin. Upon combination treatment with L-arginine and pyocyanin, however, the resulting green fluorescence decreased since there was no NO available in the system to react with superoxide and produce peroxynitrite (FIG. 3B). Alternatively, NAC treatment (5 mM) attenuated ROS induction by pyocyanin, thereby leaving available NO in the cells treated with both agents. Interestingly, general levels of NO production were increased in the NAC pretreated cells, most likely because of the suppression of superoxide production, thus preventing the scavenging of NO by superoxide (FIG. 3C).

To confirm the levels of peroxynitrite production, parallel samples were pretreated with 20 μM ebselen, specific peroxynitrite scavenger (FIG. 3D). This treatment inactivated peroxynitrite upon its production, but it did not restore the original levels of NO or superoxide in the system. Therefore, one was still able to detect the decreased levels of NO and superoxide in the combination treated sample. Green fluorescent signal decreased significantly in the case, however, where peroxynitrite was made in the system.

### Example 2

### Specific Profiling of ROS/RNS Induced in HeLa Cells by Different ROS Inducers Using Wide-Field Fluorescent Microscopy

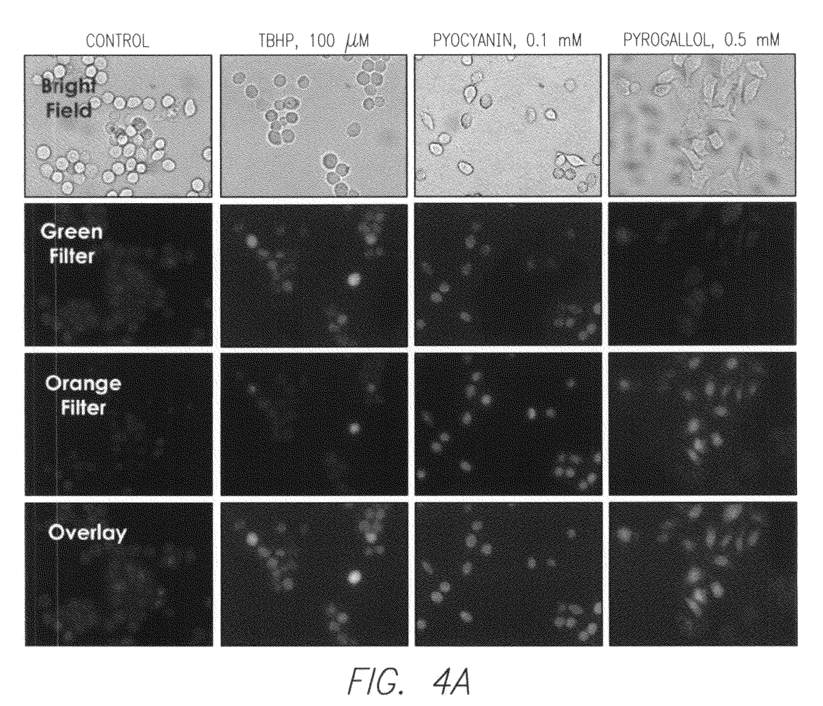

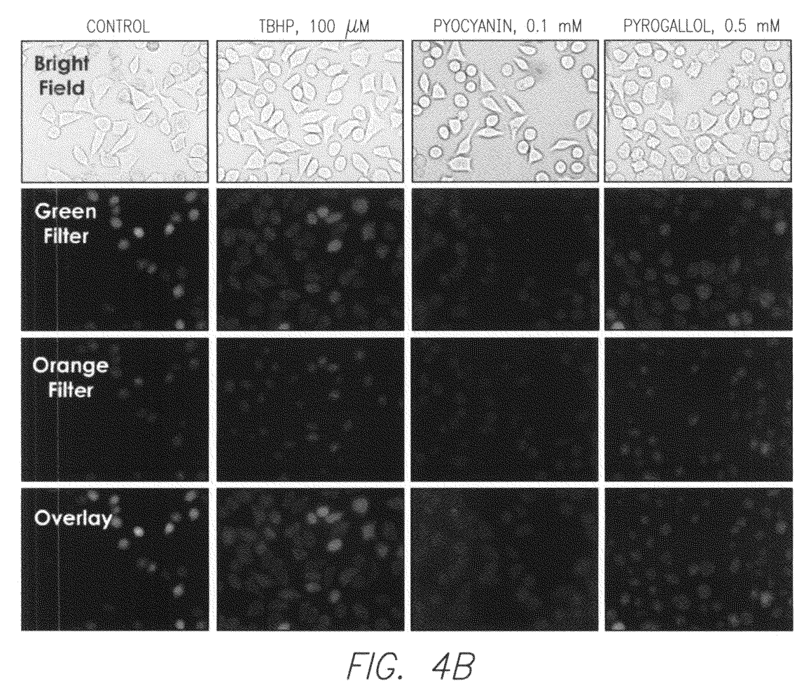

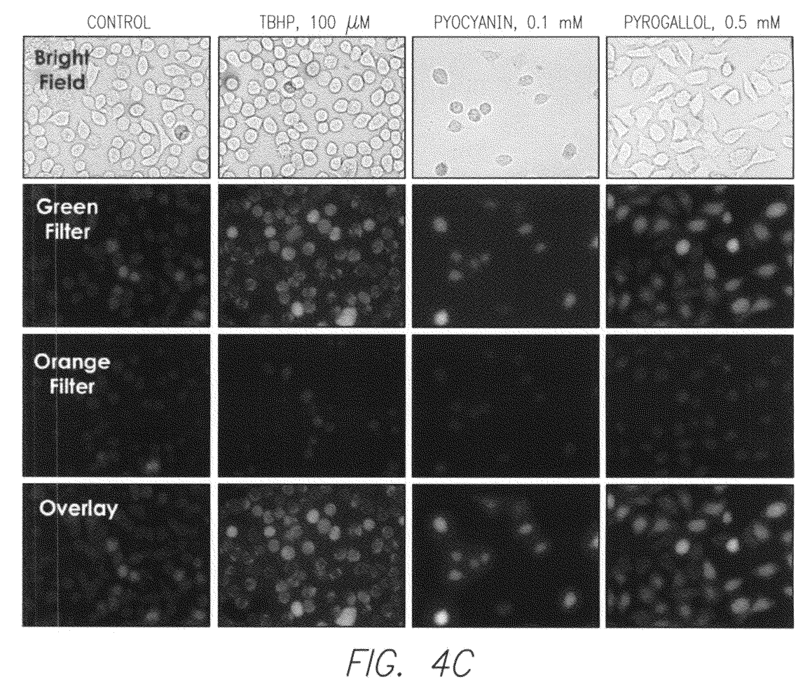

For purposes of simplifying the assay description, this example was carried out with only two indicator probes, though analogous procedures were employed as in the case where three fluorophores were utilized. Human cervical carcinoma cell line HeLa was cultured as described in Example 1. The day before the experiment, the cells were seeded in multi-well microscope slides (Cel-Line™, Portsmouth, N.H.) at a density of 2×10⁴ cells per well. On the next day, the cells were treated with different ROS inducers (0.1 mM tert-butyl hydroperoxide [TBHP], 0.1 mM pyocyanin or 0.1 mM pyrogallol) for 1 h at 37° C. After a brief wash with PBS, the cells were stained with 1 μM of DCFDA and HE in culture medium for 30 min, 37° C., washed twice with PBS, overlaid with a cover slip and observed under the fluorescent microscope, using green and orange filters described in the Table 2 (FIG. 4A). To perform specific profiling of ROS/RNS, parallel samples of HeLa cells were pretreated for 30 min with 5 mM NAC (general ROS scavenger, FIG. 4B), 5 mM Tiron (specific superoxide scavenger, FIG. 4C), 10 mM pyruvate (specific peroxide scavenger, FIG. 4D). Pretreated cells were induced as described, overlaid with a cover slip and observed under fluorescent microscope using the same set of filters.

According to the data presented on FIG. 4A, 0.5 mM pyrogallol induced mostly superoxide production in HeLa cells, while 0.1 mM of pyocyanin and 0.1 mM of tert-butyl hydroperoxide (TBHP) induced production of different ROS types, with the majority of superoxide for pyocyanin and the majority of peroxide/hydroxyl radicals/peroxynitrite for TBHP. Pretreatment with the general ROS scavenger abolished the production of all ROS types (FIG. 4B), while Tiron pretreatment attenuated only orange fluorescence in all treated cells (FIG. 4C). Interestingly, green fluorescence was increased in pyrogallol-treated cells upon pre-incubation with Tiron. This could be a consequence of pyrogallol-induced changes of Ca²⁺ homeostasis in the cells in conjunction with superoxide suppression or non-specific effects of Tiron as well (additional experiments are needed to clarify this issue). It is important that for each inhibitor/inducer pair, the effective concentrations of both inducer and inhibitor should be established particularly for the studied system. Pyruvate pretreatment abolished green fluorescence completely in pyrogallol-treated sample of HeLa cells (FIG. 4D). A certain level of green fluorescence is still present in TBHP- and pyocyanin-treated samples of HeLa cells, however, that might indicate peroxynitrite and/or hydroxyl radical presence.

It should be appreciated by those skilled in the art that ebselen (a specific peroxynitrite scavenger) could be used in combination with the foregoing scavengers. For example, 20 μM ebselen pretreatment will eliminate peroxynitrite production resulting in bright green staining.

The present invention aids in resolving the cited ambiguity in interpreting results obtained using batteries of inducers and inhibitors. Also, using three or more indicator probes in the context of ROS/RNS profiling reduced the total number of different activators and inhibitors required to comprehensively characterize a biological system.

### Example 3

### Monitoring Kinetic Changes in Levels of NO and ROS in HeLa Cells by Wide-Field Fluorescence Microscopy

HeLa cells were cultured and plated as described in Example 1. On the day of the experiment, cells were loaded with 50 μM of DAQ, 1 μM of DCFDA and HE for 2 h, 37° C. and induced with different ROS and NO inducers (1 μM of A23187, 0.2 mM of antimycin A, 1 mM of L-arginine, 0.1 mM of pyocyanin or combination of L-arginine and pyocyanin) at 37° C. Samples for fluorescent microscopy were prepared after 10, 20, 30, 45 and 60 min of treatment as described in Example 1 and analyzed using an Olympus wide field fluorescent microscope (set of filters as described in the Table 2).

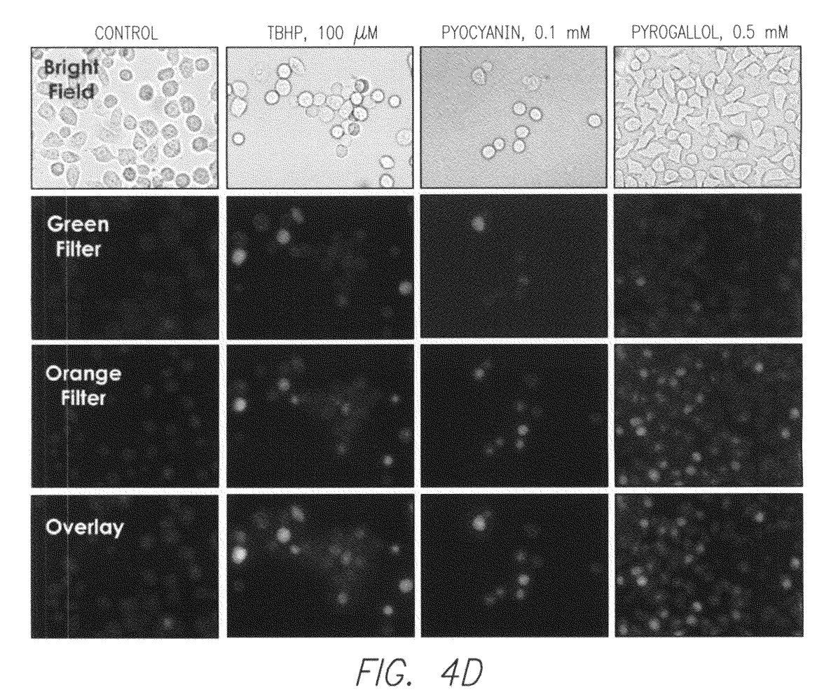

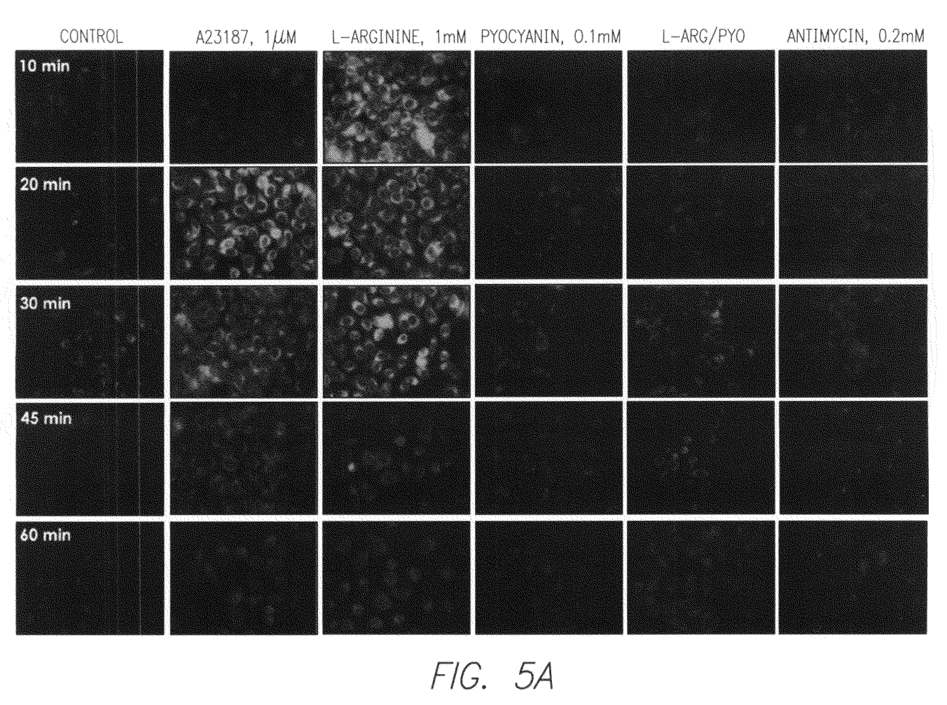

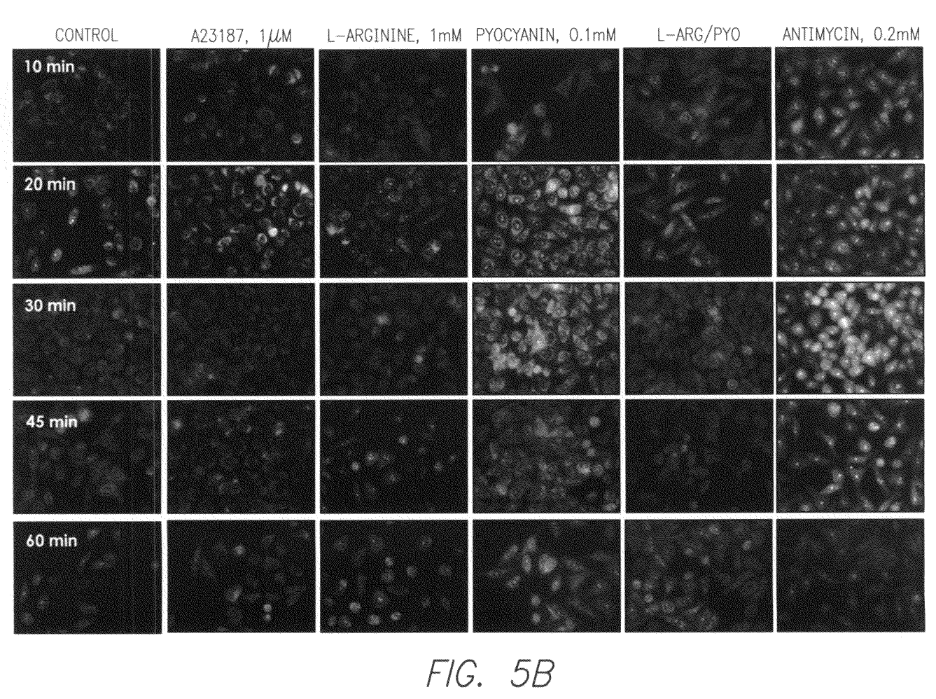

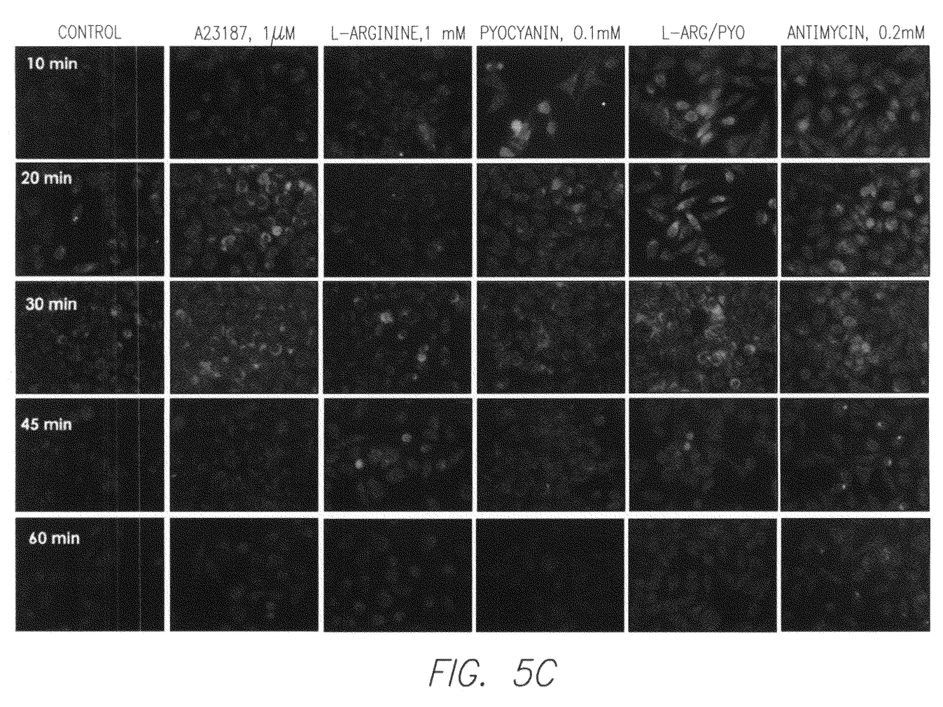

Data presented in FIG. 5, demonstrated that the developed protocol allowed real-time detection of changes in NO levels (Panel A), total ROS/RNS levels (Panel B) and in the levels of superoxide production (Panel C). L-arginine treatment quickly induced nitric oxide synthase (NOS) in HeLa cells resulting in the high levels of NO production that was detectable using DAQ as early as 10 min after the treatment (FIG. 5A). Calcium ionophore A23187 (considered as an inducer of low levels of NO) treatment resulted in detectable signal 20 min after the treatment. The intensity of the signal tended to decrease over time (probably because of the further oxidation of the fluorescent triazole product in the reductive cellular environment). Both ROS inducers, pyocyanin and antimycin A did not induce any DAQ-detectable signal. Combination treatment with L-arginine and pyocyanin did not result in significant NO signal because of the fast reaction between superoxide (induced by pyocyanin) and NO (induced by L-arginine) resulting in peroxynitrite production (detected by DCFH using green filter) (see next paragraph).

Results presented on FIGS. 5B and 5C demonstrated early induction of ROS with both pyocyanin and antimycin A. No significant ROS production was detected after the treatment of HeLa cells with L-arginine. Again, because of the peroxynitrite production from NO and superoxide, in the case of the combined treatment with L-arginine and pyocyanin, the levels of green signal (peroxides/hydroxyl radicals/peroxynitrite) was higher than those for pyocyanin treatment alone, and the levels of orange signal (superoxide) was lower.

### Example 4

### Multiplexed ROS/RNS Detection in HeLa Cells by Flow Cytometry

HeLa cells were cultured as described in Example 1. The day before the experiment, the cells were seeded in 6-well tissue culture dishes at a density of 5×10⁵ cells per well. On the day of the experiment, the cells were loaded with 50 μM of DAQ, 1 μM of DCFDA and HE (solution in culture medium) for 2 h, 37° C. and induced with L-arginine, pyocyanin or their combination, as described in Example 1. To confirm specificity and selectivity of the staining, parallel samples were treated with NAC (general ROC inhibitor) and cPTIO (general NO scavenger and NOS inhibitor). After one hour treatment, the cells were washed with PBS, trypsinized and resuspended in 0.5 ml of PBS. After resuspension, the cells were immediately analyzed by flow cytometry using FACS Calibur instrument (or any benchtop cytometer equipped with blue and red lasers could be used). Green fluorescence of oxidized DCF was detected in the FL1 channel (excitation with 488 nm blue laser, emission detection with 530/30 BP filter), red fluorescence of DHE was detected in the FL2 channel (excitation with 488 nm blue laser, emission detection with 585/42 BP filter). Fluorescence of oxidized DAQ product was detected in the FL4 channel (excitation with 635 nm red laser, emission detection with 670 LP filter). There was substantial overlap between the oxidized dye spectra; therefore, compensation was required. For compensation purposes, singly stained samples were prepared and compensation was performed using standard protocols.

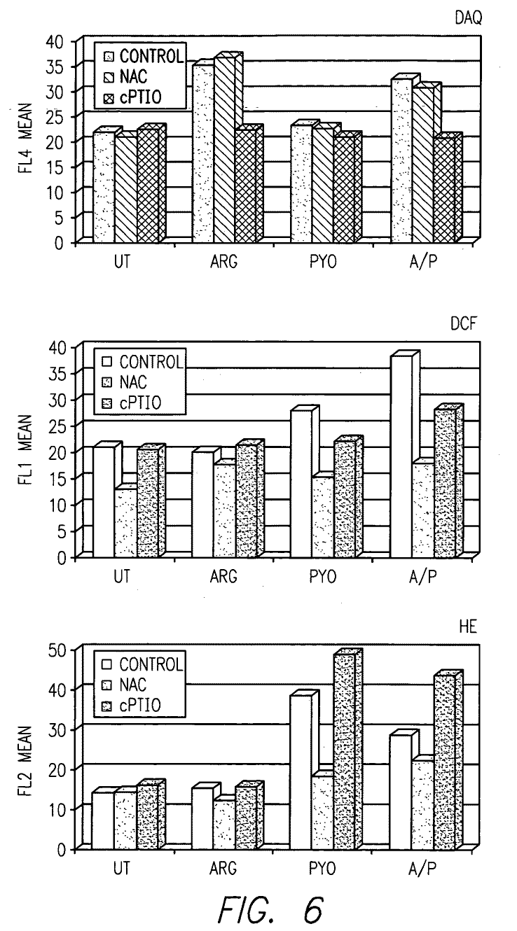

The results of the experiment are presented in FIG. 6 as a bar graph. The results obtained using flow cytometry method, correlated highly with the results of fluorescent microscopy in this system. L-arginine treatment induced NO production in HeLa cells that was detected by the fluorescence of oxidized DAQ product in FL4. This signal was blocked by pretreatment with cPTIO (NO scavenger and non-specific NOS inhibitor) but not with general antioxidant NAC pretreatment (FIG. 6, top panel). Although it did not induce significant NO production in Hela cells, pyocyanin treatment induced significant ROS production detected both in FL1 and FL2 channels (FIG. 6, middle and bottom panels) that was blocked by NAC pretreatment. Combination treatment with L-arginine and pyocyanin resulted in lower DAQ signal than after single L-arginine treatment (FIG. 6, top panel), lower superoxide signal (FIG. 6, bottom panel) but higher DCF signal than after single pyocyanin treatment (FIG. 6, middle panel). These changes in ROS/RNS profile reflected peroxynitrite production from NO and superoxide as described earlier (Example 1). The flow cytometry protocol is easily applied to a specific quantitative profiling of ROS/RNS production in live cells when the set of specific inducers and inhibitors is used (see Example 2).

### Example 5

### In Vivo Detection of ROS/RNS in Drosophila melanogaster

Direct imaging of ROS and RNS in living organisms is extremely challenging. ROS/RNS are by nature very reactive molecules and are therefore highly unstable, making it impossible to image them directly. Thus, detection of ROS/RNS levels has relied largely on detecting end products, either by chemiluminescence or by fluorescence signal that is generated when specific compounds react with them. It would be advantageous to be able to detect real time ROS/RNS production in live tissues, especially in Drosophila where the extensive genetic tools available make it possible to compare the phenotype of mutant tissue juxtaposed to its wild-type neighbor. While a protocol has been developed for imaging ROS production in Drosophila using either DCFH or DHE individually, none exist involving comprehensive three-color analysis of ROS/RNS using the combination of DCFH, DHE and DAQ.

In order to accomplish this, adult flies/larvae are first prepared for dissection It is advisable to set up crosses in such a way as to reduce crowding as much as possible. In addition, since any data obtained represents a snap shot of the rate of ROS production, it is important that larvae or adults (depending on tissue to be examined) are well fed to ensure that they are respiring optimally.

Stock solutions of DCFH, DHE and DAQ are prepared. All dyes should be reconstituted using only anhydrous solvents such as DMF or DMSO (DMF is a better choice, however, because DMSO is a hydroxyl radical scavenger itself. The anhydrous DMF can be aliquoted into 1 ml portions and kept in a dessicator. Stock solutions should be prepared immediately before use and used preferably for one batch of experiments. Make a 5 mM stock solution of DCFH, a 5 mM stock solution of DHE and a 20 mM stock of DAQ.

Larvae of the right developmental stage are collected with a paintbrush and put in phosphate-buffered saline (PBS) in three well plates, at room temperature. Alternatively for adult tissue like the germarium, females of the right age are anaesthetized and collected in 2 ml eppendorf tubes. It is important not use ice-cold PBS, as this may inhibit respiration and thus interfere with ROS production. The tissue of interest is dissected away in 1×PBS in three well glass plates. Culture medium containing amino acids should be avoided since primary amines can induce extracellular hydrolysis of the dye. In addition, it is important to remove as much extraneous tissue as possible. For instance, for third instar eye discs, the brain and salivary glands should be removed at this stage, leaving only the mouth hooks for easy transfer. This will speed up the mounting process. Delays in mounting will compromise image quality. Imaging ROS/RNS production is accomplished as follows. Reconstitute the dye right after dissection and immediately before use in anhydrous DMF. Dissolve two microliters of the reconstituted DCFH and HE dyes and five microliters of reconstituted DAQ in 1 ml of 1×PBS to give a final concentration of 10 μM for DCFH and HE and 100 μM for DAQ. Vortex to evenly disperse the dyes. Vortexing for about 15 to 30 seconds is usually optimal. Excessive vortexing may hasten decomposition of the dye, as it is subject to hydrolysis; on the other hand, shorter vortexing times may result in incomplete dispersion of the dye, resulting in the deposition of colloids on the tissue. Incubate the tissue with the dye for 5 to 15 minutes in a dark chamber, on an orbital shaker at room temperature. Then, perform three 5-minute washes in 1×PBS on an orbital shaker at room temperature. Samples should be mounted immediately in Vectashield or similar mounting medium. Images should be captured immediately using a confocal microscope. Monitoring ROS/RNS production in the wild type germarium reveals that this protocol is sensitive enough to discriminate between different levels of ROS/RNS production between different cell types of the same tissue.

Many obvious variations will no doubt be suggested to those of ordinary skill in the art in light of the above detailed description and examples of the present invention. All such variations are fully embraced by the scope and spirit of the invention as more particularly defined in the claims that now follow.

1. A method for profiling the status of reactive oxygen species (ROS), reactive nitrogen species (RNS) or reactive halogen species (RHS), and combinations thereof, in living cells or subcellular organelles, or both, said method comprising the steps of:
(A) providing:
(i) at least one sample of said living cells or cellular organelles, or both, for profiling; and
(ii) three or more indicator probes capable of providing signals, said indicator probes being independently selected from:
(a) global reactive species probes for detecting or quantifying in living cells or subcellular organelles oxidative stress, nitrative stress, or halogenating stress, and combinations thereof; and
(b) selective reactive species probes for detecting specific ROS species, specific RNS species, or specific RHS species, and combinations thereof;
(B) initially contacting said sample of living cells or subcellular organelles (i) with said three or more indicator probes (ii) to generate signals; and
(C) measuring said signals generated in step (B), thereby providing a profile status of said reactive species in said living cells or subcellular organelles, or both.

2. The method of claim 1, wherein said living cells are contained in tissue, an organ or an organism.

3. The method of claim 1, wherein said subcellular organelles comprise mitochondria, peroxisomes, cytosol, vesicles, lysosomes, plasma membranes, chloroplasts, nuclei, nucleoli, inner mitochondrial matrices, inner mitochondrial membranes, intermembrane spaces, outer mitochondrial membranes, secretory vesicles, endoplasmic reticuli, golgi bodies, phagosomes, endosomes, exosomes, plasma membranes, microtubules, microfilaments, intermediate filaments, filopodia, ruffles, lamellipodia, sarcomeres, focal contacts, podosomes, ribosomes, microsomes, lipid rafts, nuclear membranes, chloroplasts or cell walls, and combinations thereof.

4-5. (canceled)

6. The method of claim 1, wherein said reactive oxygen species (ROS) are selected from superoxide (O₂^{•-}), hydroperoxy (HO^{•}₂), hydrogen peroxide (H₂O₂), peroxynitrite (ONOO⁻), hypochlorous acid (⁻OHCl), hypobromous acid (⁻OHBr), hydroxyl radical (HO^{•}), peroxy radical (ROO^{•}), alkoxy radical (RO^{•}), singlet oxygen (¹O₂), lipid peroxides, lipid peroxyradicals or lipid alkoxyl radicals, and combinations thereof.

7. The method of claim 1, wherein said reactive nitrogen species (RNS) are selected from nitric oxide (NO), nitrogen dioxide radical (^{•}NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) or dinitrogen trioxide (N₂O₃), and combinations thereof.

8-14. (canceled)

15. The method of claim 1, wherein said providing step (A) (ii), the global reactive species probes comprise DCFDA, dihydrorhodamine 123 (DHR), C₁₁-BODIPY, DAF-2, DAR-4M, dihydrocalcein or a Redox-sensitive Green Fluorescent Protein (roGFP), 5-(and -6)-chloromethyl-2′,7′-dichlorodihydrofluorescein diacetate, acetyl ester or ABTS, and combinations thereof.

16. The method of claim 1, wherein said providing step (A) (ii), the selective reactive species probes comprise 2-(2-pyridyl)-benzothiazoline, Amplex Red, APF, Bis-2,4-dinitrobenzenesulfonyl fluoressceins, BODIPY FL EDA, CCA/SECCA, copper (II) fluorescein, CsPA (cis-parinaric acid), DAC (diaminocyanine), DAMBO-P^{H}, DAQ, DHE, DMA, DMAX, Dobz derivatives, DPAX (9-[2-(3-carboxyl-9,10-diphenyl)anthryl]-6-hydroxy-3H-xanthen-3-one), DPBF (1,3-diphenylisobenzofuran), DPPEA-HC, DPPEC, DPPP (diphenyl-1-pyrenylphosphine), FL₅, HKOCl-1, homovanilic acid, HPF, HySOX, metal-based turn-on fluoresecent probes, MitoPY1, Mito-SOX, MitoTracker Orange (dihydrotetramethyl-rosamine), NBD-Cl (4-chloro-7-nitrobenzo-2-oxa-1,3-diazole), NFDS-1, pentafluorobenzene-sulfonyl fluorescein, Peroxifluor-1, Peroxycrimson-1, Peroxygreen-1, Peroxyresorufin-1, o-Phenylenediamine derivatives, scopoletin, Spy-HP, Rhodamine spirolactam, SNAPF, Singlet Oxygen Sensor Green, Terephtalic acid and TMDA BODIPY, a selective Redox-sensitive Green Fluorescent Protein (roGFP) or HyPer, and combinations thereof.

17. (canceled)

18. The method of claim 1, wherein said providing step (A), there are further provided either:
(iii) (a) one or more inhibitors or scavengers of reactive species generation selected from ROS, RNS, RHS, and combinations thereof,
or
(iii) (b) one or more activators, donors or generators of reactive species generation selected from ROS, RNS, RHS, and combinations thereof,
or both
a combination of one or more inhibitors or scavengers (iii) (a) and one or more activators, donors or generators (iii) (b);
and wherein said contacting step (B) is carried out by contacting the living cells or subcellular organelles, or both, with said three or more indicator probes and either said one or more inhibitors or scavengers (iii) (a), said one or more activators, donors or generators (iii) (b), or both said one or more inhibitors or scavengers (iii) (a) and said one or more activators, donors or generators (iii) (b).

19-22. (canceled)

23. The method of claim 22, wherein said free-radical scavengers comprise Ebselen, mannitol, N-acetyl cysteine, pyruvate, Tiron or EUK, and combinations thereof.

24. The method of claim 18, wherein said providing step (A), the one or more activators, donors or generators (iii) (b) comprise NONOate, GEA, L-arginine, NOC, SIN-1, SNAP, sodium nitroprusside or free-radical donors/generators, and combinations thereof.

25. The method of claim 24, wherein said free-radical donors/generators comprise Antimycin A, pyocyanin, pyrogallol, PMA or TBHP, and combinations thereof.

26-28. (canceled)

29. A method for profiling the status of reactive oxygen species (ROS), reactive nitrogen species (RNS), or reactive halogen species (RHS), and combinations thereof, in living cells or subcellular organelles, or both, said method comprising the steps of:
(A) providing:
(i) at least one sample of said living cells or cellular organelles, or both, for profiling:
(ii) three or more indicator probes independently selected from:
(a) global reactive species probes for detecting or quantifying in living cells or subcellular organelles, or both, oxidative stress, nitrative stress, or halogenating stress, and combinations thereof;
(b) selective reactive species probes for detecting ROS species, RNS species or RHS species, and combinations thereof;
(iii) (a) one or more inhibitors or scavengers of reactive species generation selected from ROS, RNS or RHS, and combinations thereof, and optionally,
(b) one or more activators, donors or generators of reactive species generation selected from ROS, RNS or RHS or combinations thereof;
(B) initially contacting said sample of living cells or subcellular organelles (i), or both, with said three or more indicator probes to generate signals; and
(C) measuring said signals generated in step (B), thereby providing a status profile of said reactive species in said living cells or subcellular organelles, or both.

30-55. (canceled)

56. A kit for profiling the status of reactive oxygen species (ROS), reactive nitrogen species (RNS), or reactive halogen species (RHS), and combinations thereof, in living cells or subcellular organelles, or both, the kit comprising in packaged combination:
(i) three or more indicator probes independently selected from:
(a) global reactive species probes for detecting or quantifying in living cells or subcellular organelles oxidative stress, nitrative stress, or halogenating stress, and combinations thereof; and
(b) selective reactive species probes for detecting specific ROS species, specific RNS species or specific RHS species, and combinations thereof;
(ii) buffers; and
(iii) instructions therefor.

57. The method of claim 56, wherein said reactive oxygen species (ROS) are selected from superoxide (O₂^{•-}), hydroperoxy (HO^{•}₂), hydrogen peroxide (H₂O₂), peroxynitrite (ONOO⁻), hypochlorous acid (⁻OHCl), hypobromous acid (⁻OHBr), hydroxyl radical (HO^{•}), peroxy radical (ROO^{•}), alkoxy radical (RO^{•}), singlet oxygen (¹O₂), lipid peroxides, lipid peroxyradicals, or lipid alkoxyl radicals, and combinations thereof.

58. The method of claim 56, wherein said reactive nitrogen species (RNS) are selected from nitric oxide (NO), nitrogen dioxide radical (^{•}NO₂), peroxynitrite anion (ONOO⁻), peroxynitrous acid (ONOOH), nitrosoperoxycarbonate anion (ONOOCO₂⁻), nitronium cation (NO₂⁺), nitrosonium cation (NO⁺) or dinitrogen trioxide (N₂O₃), and combinations thereof.

59-85. (canceled)
